SEQUENCE LISTING (3 sequences)

SEQ ID NO: 1 - DNA, 48 bp, Artificial Sequence
  Synthetic Talen Pair
tcaggggcaaggagactgcactgatgcccaattggtatgtcccagacc

SEQ ID NO: 2 - DNA, 90 bp, Artificial Sequence
  Synthetic KO Pig
acaaacgtcggagtcatgttcaggggcaaggagactgcactgtaagcttgcccaattggtatgtcccagaccagtgtctggctgagttct

SEQ ID NO: 3 - DNA, 59 bp, Artificial Sequence
  Synthetic FAH Transfection sequence
atgttcaggggcaaggagactgcactgtaagcttgcccaattggtatgtcccagaccag

(19) United States
(12) Patent Application Publication
(10) Pub. No.: US 2021/0037797 A1
(43) Pub. Date: Feb. 11, 2021

(54) INDUCIBLE DISEASE MODELS METHODS OF MAKING THEM AND USE IN TISSUE COMPLEMENTATION

(71) Applicant: Recombinetics, Inc., Eagan, MN (US)

(72) Inventors: Daniel F. Carlson, Woodbury, MN (US); Cheryl Lancto, Circle Pines, MN (US); Colin Fairman, St. Paul, MN (US)

(21) Appl. No.: 16/908,947

(22) Filed: Jun. 23, 2020

Related U.S. Application Data
(63) Continuation of application No. 16/101,295, filed on Aug. 10, 2018, now abandoned.
(60) Provisional application No. 62/544,620, filed on Aug. 11, 2017.

(51) Int. Cl. A01K 67/027; C12N 15/873; C12N 5/073; C12N 5/0735; C12N 15/85

(52) U.S. Cl. CPC A01K 67/0278; C12N 2015/8527; A01K 67/0275; A01K 67/0273; A01K 67/0276; A01K 67/0271; C12N 5/0604; C12N 5/0606; C12N 15/8509; A01K 2267/025; A01K 2217/075; A01K 2217/15; A01K 2217/072; A01K 2217/206; A01K 2217/203; A01K 2227/108; C12N 2015/8518; C12N 15/873

(57) ABSTRACT

Disclosed herein, are inducible immunodeficient animals and methods to make them by adding an IL2Rg/RAG2 rescue cassette (RG-reg) or an IL2Rg/RAG2/FAH rescue cassette (FRG-reg) to a line of IL2Rg/RAG2 knockout (RG-KO) or IL2Rg/RAG2/FAH knockout (FRG-KO) swine. The rescue cassette enables line breeding of immunocompetent (regRG-KO) or (regFRG-KO) swine for rapid propagation. The rescue cassette can be excised, specifically in germ cells of regRG-KO or regFRG-KO swine, such that offspring of animals do not possess the rescue cassette and are immunodeficient. The immunodeficient swine also provide host embryos having genetic ablations to provide a niche for organ complementation by human stem cells.

### CROSS REFERENCE TO RELATED APPLICATIONS

This application claims priority to U.S. Provisional Application No. 62/544,620 filed Aug. 11, 2017 which is hereby incorporated by reference in its entirety.

### FIELD OF THE INVENTION

This invention is directed to livestock animals having introduced into their genome an inducible cassette suitable for rescue of gene knockout phenotypes and providing for increased breeding potential of genotypes that present as failure to thrive.

### BACKGROUND OF THE INVENTION

Various diseases present as failure to thrive (FTT) phenotypes and/or result in greatly decreased ability to reach maturity. One example is severe combined immunodeficiency, (SCID). SCID is a rare genetic disorder characterized by the failure of the proper development of mature T cells and B cells. SCID animals are difficult to produce due to their lack of immune system and necessity to propagate in a germ-free environment. The ability of clinicians to model such diseases and identify treatment modalities is limited as animal models of such diseases are difficult to produce due to high mortality and consequent difficulty to maintain to breeding age. Thus, in many instances, the production of such animals is a singular event requiring gene editing of a primary cell or embryo to recapitulate disease alleles followed by somatic cell nuclear transfer to produce an animal, which due to both disease phenotype and cloning inefficiencies result in a very low percent of animals actually produced. Many other diseases also present the same problem of high mortality in the neonate and the inability of such animals to grow to breeding age limits their ability to produce and maintain genetically relevant colonies of animals from which to identify appropriate treatments and drugs.

In the case of SCID, immunodeficient rodents, outside of wild type strains, are the most commonly used animals in research. However, as with many rodent models of disease, rodents fail to adequately mimic human disease phenotypes and human responses to drugs. Thus, for many preclinical tests or manufacture process, a large animal, such as the pig, is desirable. However, one drawback of large animal models is their relatively small litter size (compared to rodents), time it takes to reach maturity and breeding age and the consequent cost to maintain a significant model herd from which to develop consistent treatment paradigms. In the case of SCID, various researchers have generated small cohorts of immunodeficient pigs by knockout of IL2Rg, RAG2 or both, followed by SCNT. Unfortunately, SCNT is not a sustainable production model, and rearing herds of immunodeficient swine is not feasible due to the high mortality of such individuals.

Therefore, it would be desirable to develop a method for the production of large animal models of disease in which, at least the health of the parents of the models also did not suffer FTT and or otherwise present disease phenotypes as those models which they are used to propagate but rather provide a sustainable pipeline for model that can be reared in herds.

### SUMMARY OF THE INVENTION

Thus, disclosed herein are methods to propagate large animal models of diseases by providing a founder generation (F₀) that has been genetically edited so as to express disease-causing alleles and that is healthy due to the presence of a rescue cassette introgressed into the F₀ genome. As disclosed herein, the rescue cassette includes an inducible recombinase fused to a gamete specific promoter such that the cassette can be excised from the gametes of the F₀ animals and thus provides an F₁ generation that, lacking the cassette, expresses the disease phenotype typical of the disease alleles edited into the genome. Those of skill in the art will appreciate that the F₀ generation, having a healthy phenotype does not suffer the complications previously encountered in the breeding of large animal models of disease. Those of skill in the art will appreciate that the current standard in the field is to create conventional conditional models where the rescue cassette is removed in the F1 generation (or the experimental generation) the cassette in this invention is removed in the germline of the preceding generation, eliminating the chance of mosaic distribution of cassette removal in the experimental generation. Thus, the model disclosed provides a much greater approximation of real disease conditions with much greater ease and efficiency.

Therefore, in one exemplary embodiment, disclosed herein, is a rescue cassette configured to be introgressed into a livestock animal wherein the rescue cassette comprises a germ-line specific promoter fused to an inducible recombinase and one or more genes herein the genes are homologs of native genes found in a livestock animal. In some embodiments, the genes are under the control of their native promoter. In various embodiments, the cassette is configured for the introgression into the genome of a primary cell or embryo of a livestock animal. In various embodiments, the cassette is configured such that induction of the recombinase results in excision of the rescue cassette only in the germ-line cell of animals carrying it. In some embodiments, the genes expressed in the cassette can be augmented or increased by making use of a landing pad included in the cassette or target sequences in the cassette used to introduce one or more rescue genes into the cassette to create new lines or models. In these cases, native genes are also edited to create knockouts or disease alleles that are then rescued by the genes added to the augmented rescue cassette. Addition to the cassette can be done by any method however, in some cases introduction can be made by PITCh or HITI as described below. Of course, editing of native genes is made as described using targeting endocnucleases. In embodiments, the recombinase is induced by an estrogen receptor antagonist including but not limited to tamoxifen.

In yet other exemplary embodiments, disclosed herein is a cell or embryo having introgressed in its genome a rescue cassette as disclosed above. In some embodiments, disclosed is in an animal produced from the cell or embryo disclosed. In various embodiments, the cassette is integrated into the genome at a safe-harbor locus. In still other embodiments, the cell or embryo further has one or more native genes, homologous to those in the cassette edited. In embodiments, the edits to the native genes comprise knock-outs and/or disease alleles. In various embodiments the disease alleles are humanized alleles. In yet other embodiments, the genes expressed from the cassette are from the same species as the edited genes. In embodiments, the cell is cloned, or the embryo is implanted in a surrogate mother. In various embodiments, an animal is produced. In embodiments, the edited genes are IL2Rg and/or RAG2. In yet other embodiments the edited genes are IL2Rg and/or RAG2 and/or FAH.

In still other exemplary embodiments, disclosed herein is a livestock animal comprising, in its genome a rescue cassette including an inducible recombinase driven by a tissue specific promoter. In these embodiments, the rescue cassette is expressed in a majority of the cells of the animal and the cassette expresses one or more genes edited in the animal's genome. In these embodiments the cassette includes an inducible recombinase. In still other embodiments, the tissue specific promoter is a gamete specific promoter. In various embodiments as disclosed the rescue cassette is integrated into a safe harbor locus of the animal's gene. In embodiments, the genes expressed from the rescue cassette are driven by their native promoter. In yet other embodiments one or more of the native genes of the animal are edited. In some embodiments, one or more of the edited genes comprise a niche for organ or tissue development. In some embodiments the animal is a pig a cow a goat or a sheep. In yet other embodiments, after induction, the gametes of the animal lack the cassette. In still other embodiments, disclosed is an embryo derived from male and female gametes lacking the cassette. In yet other embodiments disclosed herein is an embryo as disclosed above complemented by one or more pluripotent cells. In yet other embodiments is an organ or tissue produced from the pluripotent cells. In some embodiments the pluripotent cells are human. In still other embodiments, the animal is immunodeficient.

In still other exemplary embodiments, disclosed herein is a method of making a livestock animal model of disease comprising: editing one or more genes associated with a disease in a fibroblast or embryo of an animal; integrating into the fibroblast or embryo genome a rescue cassette comprising: one or more of the edited genes; an inducible recombinase under control of a tissue specific promoter; wherein the tissue specific promoter is gamete specific; inducing the recombinase, wherein the rescue cassette is excised from the gametogenic tissue; wherein the gametes of the animal do not contain the rescue cassette; wherein a female gamete is fertilized by a male gamete to provide an embryo; wherein the embryo is gestated to an animal. In various embodiments the male gametes and the female gametes have the same genetic edits. In yet other embodiments the male gametes and the female games have different genetic edits. In various embodiments the genetic edits introduce disease alleles into the genome. In some embodiments the genetic edits result in knockout of the genes. In yet other embodiments the genetic edits introduce a niche for the development of organs or tissues. In still other embodiments pluripotent cells are introduced into the embryo to complement the niche. In some embodiments, pluripotent cells are from the same species. In yet other embodiments, the pluripotent cells are human. In various exemplary embodiments, the animal is pig, goat, sheep or cow. In embodiments the embryo is further modified, comprising editing one or more further genes and, the rescue cassette of the embryo is modified to introduce one or more homologs of the one or more further genes, wherein an animal is produced from the embryo, providing an F₁ generation. In some embodiments, one or more of the edited genes comprise RAG2 and/or IL2Rg. In some embodiments the edited genes comprise those found in Table 2.

These and other features and advantages of the present disclosure will be set forth or will become more fully apparent in the description that follows and in the appended claims. The features and advantages may be realized and obtained by means of the instruments and combinations particularly pointed out in the appended claims. Furthermore, the features and advantages of the disclosure may be learned by the practice of the methods and techniques disclosed herein or will be apparent from the description, as set forth hereinafter.

### BRIEF DESCRIPTION OF THE FIGURES

FIG. 1: Characterization of adult DAZL−/− porcine testes. (A, B) Histology showing the complete absence of germ cells in DAZL−/− adult testes. The basement membrane is highlighted with a dotted line. (C) In wild-type single or paired spermatogonia (arrows) expressing UCH-L1 are restricted to localization at the basement membrane. (D) UCH-L1 labeling was not detected in adult DAZL−/− testes supporting an absence of spermatogonia.

FIG. 2: Immunohistochemical characterization of juvenile DAZL−/− porcine testes. UCH-L1 is a marker for undifferentiated, type A spermatogonia. (A) In 10 wk old wildtype testes UCH-L1 positive spermatogonia (arrows) are in contact with non-expressing cells to form a single layer surrounding the lumen of the tubules. (B) UCH-L1 labeling was not detected in 10 wk DAZL−/− testes suggesting an absence of spermatogonia. The basement membrane is highlighted with a dotted line. (C, D) Expression of the Sertoli cell marker, vimentin, is similar between the 10 wk wildtype and DAZL−/− testes.

FIG. 3: Anatomical analysis of wild-type and immune deficient piglet tissues. The heart and surrounding organs were examined in necropsies of (A) wild-type and (B) immune deficient piglets. A) Thymus clearly observed in all wild-type piglets (large arrow). B) An absence of a thymus was noted in all RG-KO piglets (large arrowhead indicating the normal anatomical position). Tissues harvested from all major organs of all animals were formalin-fixed, embedded in paraffin, sectioned and examined by H&E staining. C) Spleen section from wild-type piglet. Arrows indicate the presence of normal periarterial lymphoid sheaths (PALS) surrounding central arteries within the white pulp of the spleen. D) Spleen section of immune deficient animal. Arrowheads indicate the complete absence of PALS surrounding central arteries.

FIG. 4. Leukocyte populations present in wild-type and immune deficient piglets. Total cell populations were isolated from bone marrow (BM), spleen, circulating blood, and thymus (wild-type only) of wild-type and immune deficient piglets and analyzed by flow cytometry using antibodies to specific cell markers, gating on leukocyte populations. Data is presented as the percent of total leukocyte population. The thymus was not present in immune deficient piglets. Therefore, data was reported as “not determined” (ND) for these samples.

FIG. 5: FAH transfection into RAG2/IL2Rg deficient cells. A) Pooled cell extracts showing presence of FAH unique restriction (HINDIII) site. B) Interindividual colonies. C) Schematic of strategy for FAH editing. D) Identification of positive colonies.

FIG. 6: Development and implementation of regRG-KO swine. A) Schematic of the RG-reg cassette. B) RegRG-KO swine can be propagated in standard housing prior to switching off the rescue cassette in germ cells by Tamoxifen administration in C. C) Only offspring of Tamoxifen treated are immunodeficient.

FIG. 7: The RG-reg cassette. A) RG-Reg provides rescue cassettes for Rag2 and IL2RG which makes Rag2 −/− and IL2Rg −/− pigs that carry the RG-Reg cassette immunocompetent and capable of being raised under normal rearing conditions. B) Offspring of tamoxifen treated RG-Reg pigs will no longer have Rag2-IL2Rg rescue cassette making them immunocompromised.

FIG. 8: Sus scrofa Rag2 Cassette (ssRag2). Assembly of promoter and non-coding sequence, Rag2 coding sequence OR GFP, and 3′ non-coding sequence and poly(A) signal. Gibson Assembly or traditional restriction endonuclease. GFP version will be placed into Sleeping Beauty transposon for testing in cells. To produce immunocompromised offspring, adults will be treated with tamoxifen to stimulate Cre activity in germ cells.

FIG. 9: Sus scrofa IL2RgCassette (ssILRg). Assembly of promoter and non-coding sequence, IL2Rg coding sequence OR RFP, and 3′ non-coding sequence and poly(A) signal. Gibson Assembly or traditional restriction endonuclease. RFP version will be placed into Sleeping Beauty transposon for testing in cells.

FIG. 10: DAZL-Cre-ER2 Cassette. Assembly of DAZL promoter and Cre-ER2 OR YFP-Cre. Gibson Assembly or traditional restriction endonuclease. YFP-Cre version will be placed into Sleeping Beauty transposon for testing in cells.

### DETAILED DESCRIPTION OF THE EXEMPLARY EMBODIMENTS

Provided herein are large animal models of disease and methods to propagate them. In addition, one disease model provided herein includes severe combined immunodeficiency (SCID), which provides an ideal background in which to create genetic niches for the complementation of genes providing for the development of organs and tissues. Disease models are created by editing genes in the animal's genome to convert native genes to disease causing alleles or knockouts. The animal is rescued by introgression, into a safe harbor locus, a rescue cassette expressing the edited genes and also including an inducible recombinase under the control of a tissue specific promoter such as a DAZL promoter, a VASA promoter or a NANOS promoter which are specific to gameteogenesis. Thus, induction of the recombinase results in gametes expressing the genes in edited form.

Practical applications can be found, for example, in regenerative medicine, swine can provide particular benefits with two primary goals. 1) To develop better large animal models of human disease for preclinical testing by gene editing. All novel therapies in regenerative medicine, pharmaceuticals, and medical devices are required to demonstrate safety and efficacy in animal models prior to entering human trials. Heavy reliance on rodent preclinical models has resulted in inflated failure rates due to vast differences in size, anatomy and physiology compared to humans. Pigs are widely considered the best large animal model of humans, and one goal is to develop lines of pigs that precisely mimic the human disease state leading to more relevant preclinical testing and reduced risk/cost associated with human clinical trials. 2) Engineer in vivo niches into swine to enable manufacturing of personalized human cells, tissues, and organs for research or transplantation. Immunodeficient swine serve both of these objectives in a variety of ways. First, an immunodeficient pig will allow direct assessment of human cell-based therapies in a large animal that will not reject the graft. In combination with other gene-edited lines of human disease, congenital heart failure, polycystic kidney disease etc. as examples, would allow safety and efficacy testing in the large animal model with human stem cells prepared using established clinical protocols. Together with additional mutations, in vivo niches for complementation of organs and tissues can be created.

Unless defined otherwise, all technical and scientific terms used herein have the same meanings as commonly understood by one of ordinary skill in the art to which this disclosure belongs. All publications and patents specifically mentioned herein are incorporated by reference for all purposes including describing and disclosing the chemicals, instruments, statistical analyses and methodologies which are reported in the publications which might be used in connection with the disclosure. All references cited in this specification are to be taken as indicative of the level of skill in the art. Nothing herein is to be construed as an admission that the disclosure is not entitled to antedate such disclosure by virtue of prior invention.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is disubstituted.

As used herein, “or” should be understood to have the same meaning as “and/or” as defined above. For example, when separating a listing of items, “and/or” or “or” shall be interpreted as being inclusive, e.g., the inclusion of at least one, but also including more than one, of a number of items, and, optionally, additional unlisted items. Only terms clearly indicated to the contrary, such as “only one of or “exactly one of,” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including”, “includes”, “having”, “has”, “with”, or variants thereof, are intended to be inclusive similar to the term “comprising.”

“Additive Genetic Effects” as used herein means average individual gene effects that can be transmitted from parent to progeny.

“Allele” as used herein refers to an alternate form of a gene. It also can be thought of as variations of DNA sequence. For instance, if an animal has the genotype for a specific gene of Bb, then both B and b are alleles.

As used herein, the term “knockout” in reference to a gene or nucleotide sequence refers to cell or organism in which a gene or nucleotide sequence is made inoperative.

“DNA Marker” refers to a specific DNA variation that can be tested for association with a physical characteristic.

“Genotype” refers to the genetic makeup of an animal.

“Genotyping (DNA marker testing)” refers to the process by which an animal is tested to determine the particular alleles it is carrying for a specific genetic test.

“Simple Traits” refers to traits such as coat color and horned status and some diseases that are carried by a single gene.

“Complex Traits” refers to traits such as reproduction, growth and carcass that are controlled by numerous genes.

“Complex allele”—coding region that has more than one mutation within it. This makes it more difficult to determine the effect of a given mutation because researchers cannot be sure which mutation within the allele is causing the effect.

“Copy number variation” (CNVs) a form of structural variation—are alterations of the DNA of a genome that results in the cell having an abnormal or, for certain genes, a normal variation in the number of copies of one or more sections of the DNA. CNVs correspond to relatively large regions of the genome that have been deleted (fewer than the normal number) or duplicated (more than the normal number) on certain chromosomes. For example, the chromosome that normally has sections in order as A-B-C-D might instead have sections A-B-C- “Repetitive element” patterns of nucleic acids (DNA or RNA) that occur in multiple copies throughout the genome. Repetitive DNA was first detected because of its rapid association kinetics.

“Quantitative variation” variation measured on a continuum (e.g. height in human beings) rather than in discrete units or categories. See continuous variation. The existence of a range of phenotypes for a specific character, differing by degree rather than by distinct qualitative differences.

“Homozygous” refers to having two copies of the same allele for a single gene such as BB.

“Heterozygous” refers to having different copies of alleles for a single gene such as Bb.”

“Locus” (plural “loci”) refers to the specific locations of a maker or a gene.

“Centimorgan (Cm)” a unit of recombinant frequency for measuring genetic linkage. It is defined as the distance between chromosome positions (also termed, loci or markers) for which the expected average number of intervening chromosomal crossovers in a single generation is 0.01. It is often used to infer distance along a chromosome. It is not a true physical distance however.

“Chromosomal crossover” (“crossing over”) is the exchange of genetic material between homologous chromosomes inherited by an individual from its mother and father. Each individual has a diploid set (two homologous chromosomes, e.g., 2n) one each inherited from its mother and father. During meiosis I, the chromosomes duplicate (4n) and crossover between homologous regions of chromosomes received from the mother and father may occur resulting in new sets of genetic information within each chromosome. Meiosis I is followed by two phases of cell division resulting in four haploid (1n) gametes each carrying a unique set of genetic information. Because genetic recombination results in new gene sequences or combinations of genes, diversity is increased. Crossover usually occurs when homologous regions on homologous chromosomes break and then reconnect to the other chromosome.

“Marker Assisted Selection” (MAS) refers to the process by which DNA marker information is used to assist in making management decisions.

“Marker Panel” a combination of two or more DNA markers that are associated with a particular trait.

“Non-additive Genetic Effects” refers to effects such as dominance and epistasis. Codominance is the interaction of alleles at the same locus while epistasis is the interaction of alleles at different loci.

“Nucleotide” refers to a structural component of DNA that includes one of the four base chemicals: adenine (A), thymine (T), guanine (G), and cytosine (C).

“Phenotype” refers to the outward appearance of an animal that can be measured. Phenotypes are influenced by the genetic makeup of an animal and the environment.

“Single Nucleotide Polymorphism (SNP)” is a single nucleotide change in a DNA sequence.

“Haploid genotype” or “haplotype” refers to a combination of alleles, loci or DNA polymorphisms that are linked so as to cosegregate in a significant proportion of gametes during meiosis. The alleles of a haplotype may be in linkage disequilibrium (LD).

“Linkage disequilibrium (LD)” is the non-random association of alleles at different loci i.e. the presence of statistical associations between alleles at different loci that are different from what would be expected if alleles were independently, randomly sampled based on their individual allele frequencies. If there is no linkage disequilibrium between alleles at different loci they are said to be in linkage equilibrium.

The term “restriction fragment length polymorphism” or “RFLP” refers to any one of different DNA fragment lengths produced by restriction digestion of genomic DNA or cDNA with one or more endonuclease enzymes, wherein the fragment length varies between individuals in a population.

“Introgression” also known as “introgressive hybridization”, is the movement of a gene or allele (gene flow) from one species into the gene pool of another by the repeated backcrossing of an interspecific hybrid with one of its parent species. Purposeful introgression is a long-term process; it may take many hybrid generations before the backcrossing occurs.

“Nonmeiotic introgression” genetic introgression via introduction of a gene or allele in a diploid (non-gemetic) cell. Non-meiotic introgression does not rely on sexual reproduction and does not require backcrossing and, significantly, is carried out in a single generation. In non-meiotic introgression, an allele is introduced into a haplotype via homologous recombination. The allele may be introduced at the site of an existing allele to be edited from the genome or the allele can be introduced at any other desirable site.

As used herein the term “genetic modification” refers to is the direct manipulation of an organism's genome using biotechnology.

The terms “niche” and “genetic niche” are used interchangeable herein to refer to the absence of genes that code for a particular aspect of an organism. In some cases, the niche may be an absence of genes that code for or are responsible for the development of a tissue or organ. In other cases, the niche may be created by the absence of genes that code of a particular biochemical pathway or enzymes.

“Humanized” as used herein refers to an organ or tissue harvested from a non-human animal whose protein sequences and genetic complement are more similar to those of humans than the non-human host.

“Organ” as used herein refers to a collection of tissues joined in a structural unit to serve a common function. “Tissue” as used herein refers to a collection of similar cells that together carry out a specific function.

As used herein, the term “primary cell” are cells taken directly from living tissue and established for growth in vitro. These cells have undergone very few population doublings and are therefore more representative of the main functional component of the tissue from which they are derived in comparison to continuous (tumor or artificially immortalized) cell lines thus representing a more representative model to the in vivo state. As used herein, a “fibroblast” is a type of primary cell that can be taken by a skin or tissue punch (such as an ear punch), or from fetal material. A fibroblast is a cell type that synthesizes the extracellular matrix and collagen. Fibroblast are the most common cells of connective tissue in animals.

As used herein the phrase “precision gene editing” means a process gene modification which allows geneticists to introduce (introgress) any natural trait into any breed, in a site-specific manner without the use of recombinant DNA.

“Programable Nuclease” (PNA) include zinc-finger nucleases (ZFNs), transcription activator-like effector nucleases (TALENs) and RNA-guided engineered nucleases (RGENs) derived from the bacterial clustered regularly interspaced short palindromic repeat (CRISPR)—Cas (CRISPR-associated) system—enable targeted genetic modifications in cultured cells, as well as in whole animals and plants. These enzymes induce site-specific DNA cleavage in the genome, the repair (through endogenous mechanisms) of which allows high-precision genome editing.

“Transcription activator-like effector nucleases” (TALENs) one technology for gene editing are artificial restriction enzymes generated by fusing a TAL effector DNA-binding domain to a DNA cleavage domain.

“Zinc finger nucleases” (ZFNs) as used herein are another technology useful for gene editing and are a class of engineered DNA-binding proteins that facilitate targeted editing of the genome by creating double-strand breaks in DNA at user-specified locations.

“Meganuclease” as used herein are another technology useful for gene editing and are endodeoxyribonucleases characterized by a large recognition site (double-stranded DNA sequences of 12 to 40 base pairs); as a result, this site generally occurs only once in any given genome. For example, the 18-base pair sequence recognized by the I-SceI meganuclease would on average require a genome twenty times the size of the human genome to be found once by chance (although sequences with a single mismatch occur about three times per human-sized genome). Meganucleases are therefore considered to be the most specific naturally occurring restriction enzymes.

“CRISPR/CAS” technology as used herein refers to “CRISPRs” (clustered regularly interspaced short palindromic repeats), segments of prokaryotic DNA containing short repetitions of base sequences. Each repetition is followed by short segments of “spacer DNA” from previous exposures to a bacterial virus or plasmid. “CAS” (CRISPR associated protein 9) is an RNA-guided DNA endonuclease enzyme associated with the CRISPR. By delivering the Cas9 protein and appropriate guide RNAs into a cell, the organism's genome can be cut at any desired location.

“Indel” as used herein is shorthand for “insertion” or “deletion” referring to a modification of the DNA in an organism.

As used herein the term “renucleated egg” refers to an enucleated egg used for somatic cell nuclear transfer in which the modified nucleus of a somatic cell has been introduced.

“Genetic marker” as used herein refers to a gene/allele or known DNA sequence with a known location on a chromosome. The markers may be any genetic marker e.g., one or more alleles, haplotypes, haplogroups, loci, quantitative trait loci, or DNA polymorphisms [restriction fragment length polymorphisms (RFLPs), amplified fragment length polymorphisms (AFLPs), single nuclear polymorphisms (SNPs), indels, short tandem repeats (STRs), microsatellites and minisatellites]. Conveniently, the markers are SNPs or STRs such as microsatellites, and more preferably SNPs. Preferably, the markers within each chromosome segment are in linkage disequilibrium.

As used herein, the phrase “rescue cassette” means a nucleic acid sequence having expressed sequences that save a cell or animal from a genomic edit which would otherwise be lethal or cause failure to thrive for animals reared under normal conditions. In some embodiments, the expressed sequences are copies of the genes edited. In some embodiments, the gene are under control of their native promoters and regulatory elements such that the genes are expressed as in a physiologic wild type cell or animal. In other embodiments, the genes are under the control of special promoters such as from other tissues which may be inducible, or which may be constitutive. In still other embodiments, the promoter may be tissue specific and inducible.

As used herein, the phrase “gene in a functional form” refers to a gene that may have been edited i.e., a unique restriction site may have been introduced in to the gene however the gene continues to express a product which maintains is physiologic function to a greater or lesser degree.

As used herein the term “host animal” means an animal which has a native genetic complement of a recognized species or breed of animal.

As used herein, “native haplotype” or “native genome” means the natural DNA of a particular species or breed of animal that is chosen to be the recipient of a gene or allele that is not present in the host animal.

As used herein the term “target locus” means a specific location of a known allele on a chromosome.

The term “safe harbor” or “safe harbor locus” as used herein refers to a site in a genome in which a gene or nucleotide sequence can be introduced without interrupting a native gene function and which is transcriptionally active, e.g., in which a transgene can be expected to have a consistent level of expression. Examples of safe harbor loci are the ROSA26 locus in mice (and its orthologs) and the AAVS1 locus in humans (and its orthologs).

As used herein the term “landing pad” refers to a known nucleic acid sequence inserted into genome which optimizes the further insertion of exogenous DNA.

As used herein, the term “quantitative trait” refers to a trait that fits into discrete categories. Quantitative traits occur as a continuous range of variation such as that amount of milk a particular breed can give or the length of a tail. Generally, a larger group of genes controls quantitative traits.

As used herein, the term “qualitative trait” is used to refer to a trait that falls into different categories. These categories do not have any certain order. As a general rule, qualitative traits are monogenic, meaning the trait is influenced by a single gene. Examples of qualitative traits include blood type and flower color, for example.

As used herein, the term “quantitative trait locus (QTL)” is a section of DNA (the locus) that correlates with variation in a phenotype (the quantitative trait).

As used herein the term “cloning” means production of genetically identical organisms asexually.

The term “blastocyst” is used broadly herein to refer to embryos from two cells to about three weeks.

The term “embryo” is used broadly to refer to animals from zygote to live birth.

The term “gametogenesis” means the production of haploid sex cells (ova and spermatozoa) that each carry one-half the genetic compliment of the parents from the germ cell line of each parent. The production of spermatozoa is spermatogenesis. The fusion of spermatozoa and ova during fertilization results in a zygote cell that has a diploid genome.

The term “gametogenic cell” refers to a progenitor to an ovum or sperm, typically a germ cell or a spermatogonial cell.

“Totipotent” as used herein refers to a cell that retains the ability to differentiate into all cells of an embryo as well as extraembryonic and placental cells. “Pluripotent” refers to cells that can give rise to all embryonic cells. Examples of pluripotent cells include embryonic stem cells and induced pluripotent stem cells (IPSC)

“Somatic cell nuclear transfer” (“SCNT”) is one strategy for cloning a viable embryo from a body cell and an egg cell. The technique consists of taking an enucleated oocyte (egg cell) and implanting a donor nucleus from a somatic (body) cell.

“Orthologous” as used herein refers to a gene with similar function to a gene in an evolutionarily related species. The identification of orthologues is useful for gene function prediction. In the case of livestock, orthologous genes are found throughout the animal kingdom and those found in other mammals may be particularly useful for transgenic replacement. This is particularly true for animals of the same species, breed or lineages wherein species are defined as two animals so closely related as to being able to produce fertile offspring via sexual reproduction; breed is defined as a specific group of domestic animals having homogenous phenotype, homogenous behavior and other characteristics that define the animal from others of the same species; and wherein lineage is defined as continuous line of descent; a series of organisms, populations, cells, or genes connected by ancestor/descendent relationships. For example, domesticated cattle are of two distinct lineages both arising from ancient aurochs. One lineage descends from the domestication of aurochs in the Middle East while the second distinct lineage descends from the domestication of the aurochs on the Indian subcontinent.

The terms “knockout”, “inactivated”, and “disrupted” and variants thereof are used interchangeably herein to mean that a gene expression product is eliminated, non-functional or greatly reduced, by any means, so that the gene's expression no longer has a significant impact on the animal as a whole. These terms are sometimes used elsewhere to refer to observably reducing the role of a gene without essentially eliminating its role. These terms generally refer to preventing the formation of a functional gene product. A gene product is functional only if it fulfills its normal (wild-type) functions. Disruption of the gene prevents expression of a functional factor encoded by the gene and comprises an insertion, deletion, or substitution of one or more bases in a sequence encoded by the gene and/or a promoter and/or an operator that is necessary for expression of the gene in the animal. The disrupted gene may be disrupted by, e.g., removal of at least a portion of the gene from a genome of the animal, alteration of the gene to prevent expression of a functional factor encoded by the gene, an interfering RNA, or expression of a dominant negative factor by an exogenous gene.

“Genotyping” or “genetic testing” generally refers to detecting one or more markers of interest e.g., SNPs in a sample from an individual being tested, and analyzing the results obtained to determine the haplotype of the subject. As will be apparent from the disclosure herein, it is one exemplary embodiment to detect the one or more markers of interest using a high-throughput system comprising a solid support consisting essentially of or having nucleic acids of different sequence bound directly or indirectly thereto, wherein each nucleic acid of different sequence comprises a polymorphic genetic marker derived from an ancestor or founder that is representative of the current population and, more preferably wherein said high-throughput system comprises sufficient markers to be representative of the genome of the current population. Preferred samples for genotyping comprise nucleic acid, e.g., RNA or genomic DNA and preferably genomic DNA. A breed of livestock animal can be readily established by evaluating its genetic markers.

“Gene editing” is a type of genetic engineering in which DNA is inserted, deleted or replaced in the genome of a living organism using engineered nucleases, or “molecular scissors.” These nucleases create site-specific double-strand breaks (DSBs) at desired locations in the genome. The induced double-strand breaks are repaired through nonhomologous end-joining (NHEJ) or homologous recombination (HR), resulting in targeted mutations (‘edits’).

The term “natural” or “native” allele in the context of genetic modification means an allele found in nature in the same species of organism that is being modified. The term novel allele means a non-natural allele. A human allele placed into a goat is a novel allele. The term synthetic allele means an allele that is not found in nature. Thus, a natural allele is a variation already existing within a species that can be interbred. And a novel allele is one that does not exist within a species that can be interbred. Movement of an allele interspecies means from one species of animal to another and movement intraspecies means movement between animals of the same species.

The term “proximate” as used herein means close to.

Livestock may be genotyped to identify various genetic markers. Genotyping is a term that refers to the process of determining differences in the genetic make-up (genotype) of an individual by determining the individual's DNA sequence using a biological assay and comparing it to another individual's sequence or to a reference sequence. A genetic marker is a known DNA sequence, with a known location on a chromosome; they are consistently passed on through breeding, so they can be traced through a pedigree or phylogeny. Genetic markers can be a sequence comprising a plurality of bases, or a single nucleotide polymorphism (SNP) at a known location. The breed of a livestock animal can be readily established by evaluating its genetic markers. Many markers are known and there are many different measurement techniques that attempt to correlate the markers to traits of interest, or to establish a genetic value of an animal for purposes of future breeding or expected value.

Homology Directed Repair (HDR)

Homology directed repair (HDR) is a mechanism in cells to repair ssDNA and double stranded DNA (dsDNA) lesions. This repair mechanism can be used by the cell when there is an HDR template present that has a sequence with significant homology to the lesion site. Specific binding, as that term is commonly used in the biological arts, refers to a molecule that binds to a target with a relatively high affinity compared to non-target tissues, and generally involves a plurality of non-covalent interactions, such as electrostatic interactions, van der Waals interactions, hydrogen bonding, and the like. Specific hybridization is a form of specific binding between nucleic acids that have complementary sequences. Proteins can also specifically bind to DNA, for instance, in TALENs or CRISPR/Cas9 systems or by Gal4 motifs. Introgression of an allele refers to a process of copying an exogenous allele over an endogenous allele with a template-guided process. The endogenous allele might actually be excised and replaced by an exogenous nucleic acid allele in some situations, but present theory is that the process is a copying mechanism. Since alleles are gene pairs, there is significant homology between them. The allele might be a gene that encodes a protein, or it could have other functions such as encoding a bioactive RNA chain or providing a site for receiving a regulatory protein or RNA.

The HDR template is a nucleic acid that comprises the allele that is being introgressed. The template may be a dsDNA or a single-stranded DNA (ssDNA). ssDNA templates are preferably from about 20 to about 5000 residues although other lengths can be used. Artisans will immediately appreciate that all ranges and values within the explicitly stated range are contemplated; e.g., from 500 to 1500 residues, from 20 to 100 residues, and so forth. The template may further comprise flanking sequences that provide homology to DNA adjacent to the endogenous allele or the DNA that is to be replaced. The template may also comprise a sequence that is bound to a targeted nuclease system, and is thus the cognate binding site for the system's DNA-binding member. The term cognate refers to two biomolecules that typically interact, for example, a receptor and its ligand. In the context of HDR processes, one of the biomolecules may be designed with a sequence to bind with an intended, i.e., cognate, DNA site or protein site.

### Targeted Endonuclease Systems

Genome editing tools such as transcription activator-like effector nucleases (TALENs) and zinc finger nucleases (ZFNs) have impacted the fields of biotechnology, gene therapy and functional genomic studies in many organisms. More recently, RNA-guided endonucleases (RGENs) are directed to their target sites by a complementary RNA molecule. The Cas9/CRISPR system is a REGEN. tracrRNA is another such tool. These are examples of targeted nuclease systems: these systems have a DNA-binding member that localizes the nuclease to a target site. The site is then cut by the nuclease. TALENs and ZFNs have the nuclease fused to the DNA-binding member. Cas9/CRISPR are cognates that find each other on the target DNA. The DNA-binding member has a cognate sequence in the chromosomal DNA. The DNA-binding member is typically designed in light of the intended cognate sequence so as to obtain a nucleolytic action at nor near an intended site. Certain embodiments are applicable to all such systems without limitation; including, embodiments that minimize nuclease re-cleavage, embodiments for making SNPs with precision at an intended residue, and placement of the allele that is being introgressed at the DNA-binding site.

### TALENs

The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA or a TALEN-pair.

The cipher for TALs has been reported (PCT Publication WO 2011/072246) wherein each DNA binding repeat is responsible for recognizing one base pair in the target DNA sequence. The residues may be assembled to target a DNA sequence. In brief, a target site for binding of a TALEN is determined and a fusion molecule comprising a nuclease and a series of RVDs that recognize the target site is created. Upon binding, the nuclease cleaves the DNA so that cellular repair machinery can operate to make a genetic modification at the cut ends. The term TALEN means a protein comprising a Transcription Activator-like (TAL) effector binding domain and a nuclease domain and includes monomeric TALENs that are functional per se as well as others that require dimerization with another monomeric TALEN. The dimerization can result in a homodimeric TALEN when both monomeric TALEN are identical or can result in a heterodimeric TALEN when monomeric TALEN are different. TALENs have been shown to induce gene modification in immortalized human cells by means of the two-major eukaryotic DNA repair pathways, non-homologous end joining (NHEJ) and homology directed repair. TALENs are often used in pairs but monomeric TALENs are known. Cells for treatment by TALENs (and other genetic tools) include a cultured cell, an immortalized cell, a primary cell, a primary somatic cell, a zygote, a germ cell, a primordial germ cell, a blastocyst, or a stem cell. In some embodiments, a TAL effector can be used to target other protein domains (e.g., non-nuclease protein domains) to specific nucleotide sequences. For example, a TAL effector can be linked to a protein domain from, without limitation, a DNA 20 interacting enzyme (e.g., a methylase, a topoisomerase, an integrase, a transposase, or a ligase), a transcription activators or repressor, or a protein that interacts with or modifies other proteins such as histones. Applications of such TAL effector fusions include, for example, creating or modifying epigenetic regulatory elements, making site-specific insertions, deletions, or repairs in DNA, controlling gene expression, and modifying chromatin structure.

The term nuclease includes exonucleases and endonucleases. The term endonuclease refers to any wild-type or variant enzyme capable of catalyzing the hydrolysis (cleavage) of bonds between nucleic acids within a DNA or RNA molecule, preferably a DNA molecule. Non-limiting examples of endonucleases include type II restriction endonucleases such as FokI, HhaI, HindIII, NotI, BbvC1, EcoRI, BglII, and AlwI. Endonucleases comprise also rare-cutting endonucleases when having typically a polynucleotide recognition site of about 12-45 basepairs (bp) in length, more preferably of 14-45 bp. Rare-cutting endonucleases induce DNA double-strand breaks (DSBs) at a defined locus. Rare-cutting endonucleases can for example be a targeted endonuclease, a chimeric Zinc-Finger nuclease (ZFN) resulting from the fusion of engineered zinc-finger domains with the catalytic domain of a restriction enzyme such as FokI or a chemical endonuclease. In chemical endonucleases, a chemical or peptidic cleaver is conjugated either to a polymer of nucleic acids or to another DNA recognizing a specific target sequence, thereby targeting the cleavage activity to a specific sequence. Chemical endonucleases also encompass synthetic nucleases like conjugates of orthophenanthroline, a DNA cleaving molecule, and triplex-forming oligonucleotides (TFOs), known to bind specific DNA sequences. Such chemical endonucleases are comprised in the term “endonuclease” according to the present invention. Examples of such endonuclease include I-See I, I-Chu I, I-Cre I, I-Csm I, PI-See I, PI-Tti I, PI-Mtu I, I-Ceu I, I-See IL 1-See III, HO, PI-Civ I, PI-Ctr I, PI-Aae I, PI-Bsu I, PI-Dha I, PI-Dra I, PI-May I, PI-Meh I, PI-Mfu I, PI-Mfl I, PI-Mga I, PI-Mgo I, PI-Min I, PI-Mka I, PI-Mle I, PI-Mma I, PI-30 Msh I, PI-Msm I, PI-Mth I, PI-Mtu I, PI-Mxe I, PI-Npu I, PI-Pfu I, PI-Rma I, PI-Spb I, PI-Ssp I, PI-Fae I, PI-Mja I, PI-Pho I, PI-Tag I, PI-Thy I, PI-Tko I, PI-Tsp I, I-MsoI.

A genetic modification made by TALENs or other tools may be, for example, chosen from the list consisting of an insertion, a deletion, insertion of an exogenous nucleic acid fragment, and a substitution. The term insertion is used broadly to mean either literal insertion into the chromosome or use of the exogenous sequence as a template for repair. In general, a target DNA site is identified, and a TALEN-pair is created that will specifically bind to the site. The TALEN is delivered to the cell or embryo, e.g., as a protein, mRNA or by a vector that encodes the TALEN. The TALEN cleaves the DNA to make a double-strand break that is then repaired, often resulting in the creation of an indel, or incorporating sequences or polymorphisms contained in an accompanying exogenous nucleic acid that is either inserted into the chromosome or serves as a template for repair of the break with a modified sequence. This template-driven repair is a useful process for changing a chromosome, and provides for effective changes to cellular chromosomes.

The term “exogenous nucleic acid” means a nucleic acid that is added to the cell or embryo, regardless of whether the nucleic acid is the same or distinct from nucleic acid sequences naturally in the cell. The term nucleic acid fragment is broad and includes a chromosome, expression cassette, gene, DNA, RNA, mRNA, or portion thereof. The cell or embryo may be, for instance, chosen from the group consisting non-human vertebrates, non-human primates, cattle, horse, swine, sheep, chicken, avian, rabbit, goats, dog, cat, laboratory animal, and fish.

Some embodiments involve a composition or a method of making a genetically modified livestock and/or artiodactyl comprising introducing a TALEN-pair into livestock and/or an artiodactyl cell or embryo that makes a genetic modification to DNA of the cell or embryo at a site that is specifically bound by the TALEN-pair, and producing the livestock animal/artiodactyl from the cell. Direct injection may be used for the cell or embryo, e.g., into a zygote, blastocyst, or embryo. Alternatively, the TALEN and/or other factors may be introduced into a cell using any of many known techniques for introduction of proteins, RNA, mRNA, DNA, or vectors. Genetically modified animals may be made from the embryos or cells according to known processes, e.g., implantation of the embryo into a gestational host, or various cloning methods. The phrase “a genetic modification to DNA of the cell at a site that is specifically bound by the TALEN”, or the like, means that the genetic modification is made at the site cut by the nuclease on the TALEN when the TALEN is specifically bound to its target site. The nuclease does not cut exactly where the TALEN-pair binds, but rather at a defined site between the two binding sites.

Some embodiments involve a composition or a treatment of a cell that is used for cloning the animal. The cell may be a livestock and/or artiodactyl cell, a cultured cell, a primary cell, a primary somatic cell, a zygote, a germ cell, a primordial germ cell, or a stem cell. For example, an embodiment is a composition or a method of creating a genetic modification comprising exposing a plurality of primary cells in a culture to TALEN proteins or a nucleic acid encoding a TALEN or TALENs. The TALENs may be introduced as proteins or as nucleic acid fragments, e.g., encoded by mRNA or a DNA sequence in a vector.

### Zinc Finger Nucleases

Zinc-finger nucleases (ZFNs) are artificial restriction enzymes generated by fusing a zinc finger DNA-binding domain to a DNA-cleavage domain. Zinc finger domains can be engineered to target desired DNA sequences, and this enables zinc-finger nucleases to target unique sequences within complex genomes. By taking advantage of endogenous DNA repair machinery, these reagents can be used to alter the genomes of higher organisms. ZFNs may be used in method of inactivating genes.

A zinc finger DNA-binding domain has about 30 amino acids and folds into a stable structure. Each finger primarily binds to a triplet within the DNA substrate. Amino acid residues at key positions contribute to most of the sequence-specific interactions with the DNA site. These amino acids can be changed while maintaining the remaining amino acids to preserve the necessary structure. Binding to longer DNA sequences is achieved by linking several domains in tandem. Other functionalities like non-specific FokI cleavage domain (N), transcription activator domains (A), transcription repressor domains (R) and methylases (M) can be fused to a ZFPs to form ZFNs respectively, zinc finger transcription activators (ZFA), zinc finger transcription repressors (ZFR, and zinc finger methylases (ZFM). Materials and methods for using zinc fingers and zinc finger nucleases for making genetically modified animals are disclosed in, e.g., U.S. Pat. No. 8,106,255; U.S. 2012/0192298; U.S. 2011/0023159; and U.S. 2011/0281306.

Vectors and Nucleic acids

A variety of nucleic acids may be introduced into cells, for knockout purposes, for inactivation of a gene, to obtain expression of a gene, or for other purposes. As used herein, the term nucleic acid includes DNA, RNA, and nucleic acid analogs, and nucleic acids that are double-stranded or single-stranded (i.e., a sense or an antisense single strand). Nucleic acid analogs can be modified at the base moiety, sugar moiety, or phosphate backbone to improve, for example, stability, hybridization, or solubility of the nucleic acid. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained.

The target nucleic acid sequence can be operably linked to a regulatory region such as a promoter. Regulatory regions can be porcine regulatory regions or can be from other species. As used herein, operably linked refers to positioning of a regulatory region relative to a nucleic acid sequence in such a way as to permit or facilitate transcription of the target nucleic acid.

In general, type of promoter can be operably linked to a target nucleic acid sequence. Examples of promoters include, without limitation, tissue-specific promoters, constitutive promoters, inducible promoters, and promoters responsive or unresponsive to a particular stimulus. In some embodiments, a promoter that facilitates the expression of a nucleic acid molecule without significant tissue- or temporal-specificity can be used (i.e., a constitutive promoter). For example, a beta-actin promoter such as the chicken beta-actin gene promoter, ubiquitin promoter, miniCAGs promoter, glyceraldehyde-3-phosphate dehydrogenase (GAPDH) promoter, or 3-phosphoglycerate kinase (PGK) promoter can be used, as well as viral promoters such as the herpes simplex virus thymidine kinase (HSV-TK) promoter, the SV40 promoter, or a cytomegalovirus (CMV) promoter. In some embodiments, a fusion of the chicken beta actin gene promoter and the CMV enhancer is used as a promoter. See, for example, Xu et al., Hum. Gene Ther. 12:563, 2001; and Kiwaki et al., Hum. Gene Ther. 7:821, 1996.

Additional regulatory regions that may be useful in nucleic acid constructs, include, but are not limited to, polyadenylation sequences, translation control sequences (e.g., an internal ribosome entry segment, IRES), enhancers, inducible elements, or introns. Such regulatory regions may not be necessary, although they may increase expression by affecting transcription, stability of the mRNA, translational efficiency, or the like. Such regulatory regions can be included in a nucleic acid construct as desired to obtain optimal expression of the nucleic acids in the cell(s). Sufficient expression, however, can sometimes be obtained without such additional elements.

A nucleic acid construct may be used that encodes signal peptides or selectable expressed markers. Signal peptides can be used such that an encoded polypeptide is directed to a particular cellular location (e.g., the cell surface). Non-limiting examples of selectable markers include puromycin, ganciclovir, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable trans formants in culture. Other selectable markers include fluorescent polypeptides, such as green fluorescent protein or yellow fluorescent protein.

In some embodiments, a sequence encoding a selectable marker can be flanked by recognition sequences for a recombinase such as, e.g., Cre or Flp. For example, the selectable marker can be flanked by loxP recognition sites (34-bp recognition sites recognized by the Cre recombinase) or FRT recognition sites such that the selectable marker can be excised from the construct. See, Orban et al., Proc. Natl. Acad. Sci., 89:6861, 1992, for a review of Cre/lox technology, and Brand and Dymecki, Dev. Cell, 6:7, 2004. A transposon containing a Cre- or Flp-activatable transgene interrupted by a selectable marker gene also can be used to obtain transgenic animals with conditional expression of a transgene. For example, a promoter driving expression of the marker/transgene can be either ubiquitous or tissue-specific, which would result in the ubiquitous or tissue-specific expression of the marker in F0 animals (e.g., pigs). Tissue specific activation of the transgene can be accomplished, for example, by crossing a pig that ubiquitously expresses a marker-interrupted transgene to a pig expressing Cre or Flp in a tissue-specific manner, or by crossing a pig that expresses a marker-interrupted transgene in a tissue-specific manner to a pig that ubiquitously expresses Cre or Flp recombinase. Controlled expression of the transgene or controlled excision of the marker allows expression of the transgene.

In some embodiments, the exogenous nucleic acid encodes a polypeptide. A nucleic acid sequence encoding a polypeptide can include a tag sequence that encodes a “tag” designed to facilitate subsequent manipulation of the encoded polypeptide (e.g., to facilitate localization or detection). Tag sequences can be inserted in the nucleic acid sequence encoding the polypeptide such that the encoded tag is located at either the carboxyl or amino terminus of the polypeptide. Non-limiting examples of encoded tags include glutathione S-transferase (GST) and FLAG™ tag (Kodak, New Haven, Conn.).

Nucleic acid constructs can be introduced into embryonic, fetal, or adult artiodactyl/livestock cells of any type, including, for example, germ cells such as an oocyte or an egg, a progenitor cell, an adult or embryonic stem cell, a primordial germ cell, a kidney cell such as a PK-15 cell, an islet cell, a beta cell, a liver cell, or a fibroblast such as a dermal fibroblast, using a variety of techniques. Non-limiting examples of techniques useful for introduction of nucleic acid constructs into cells and/or embryos include the use of transposon systems, recombinant viruses that can infect cells, or liposomes or other non-viral methods such as electroporation, microinjection, or calcium phosphate precipitation, that are capable of delivering nucleic acids to cells including gene targeting by HDR, “PITCh” (Precise Integration into Target Chromosomes) or “HITI” (homology-independent targeted integration).

In transposon systems, the transcriptional unit of a nucleic acid construct, i.e., the regulatory region operably linked to an exogenous nucleic acid sequence, is flanked by an inverted repeat of a transposon. Several transposon systems, including, for example, Sleeping Beauty (see, U.S. Pat. No. 6,613,752 and U.S. 2005/0003542); Frog Prince (Miskey et al., Nucleic Acids Res., 31:6873, 2003); Tol2 (Kawakami, Genome Biology, 8(Suppl.1):S7, 2007); Minos (Pavlopoulos et al., Genome Biology, 8(Suppl.1):52, 2007); Hsmar1 (Miskey et al., Mol Cell Biol., 27:4589, 2007); and Passport have been developed to introduce nucleic acids into cells, including mice, human, and pig cells. The Sleeping Beauty transposon is particularly useful. A transposase can be delivered as a protein, encoded on the same nucleic acid construct as the exogenous nucleic acid, can be introduced on a separate nucleic acid construct, or provided as an mRNA (e.g., an in vitro-transcribed and capped mRNA).

Nucleic acids can be incorporated into vectors. A vector is a broad term that includes any specific DNA segment that is designed to move from a carrier into a target DNA. A vector may be referred to as an expression vector, or a vector system, which is a set of components needed to bring about DNA insertion into a genome or other targeted DNA sequence such as an episome, plasmid, or even virus/phage DNA segment. Vector systems such as viral vectors (e.g., retroviruses, adeno-associated virus and integrating phage viruses), and non-viral vectors (e.g., transposons) used for gene delivery in animals have two basic components: 1) a vector comprised of DNA (or RNA that is reverse transcribed into a cDNA) and 2) a transposase, recombinase, or other integrase enzyme that recognizes both the vector and a DNA target sequence and inserts the vector into the target DNA sequence. Vectors most often contain one or more expression cassettes that comprise one or more expression control sequences, wherein an expression control sequence is a DNA sequence that controls and regulates the transcription and/or translation of another DNA sequence or mRNA, respectively.

Many different types of vectors are known. For example, plasmids and viral vectors, e.g., retroviral vectors, are known. Mammalian expression plasmids typically have an origin of replication, a suitable promoter and optional enhancer, and also any necessary ribosome binding sites, a polyadenylation site, splice donor and acceptor sites, transcriptional termination sequences, and 5′ flanking non-transcribed sequences. Examples of vectors include: plasmids (which may also be a carrier of another type of vector), adenovirus, adeno-associated virus (AAV), lentivirus (e.g., modified HIV-1, SIV or FIV), retrovirus (e.g., ASV, ALV or MoMLV), and transposons (e.g., Sleeping Beauty, P-elements, Tol-2, Frog Prince, piggyBac).

As used herein, the term nucleic acid refers to both RNA and DNA, including, for example, cDNA, genomic DNA, synthetic (e.g., chemically synthesized) DNA, as well as naturally occurring and chemically modified nucleic acids, e.g., synthetic bases or alternative backbones. A nucleic acid molecule can be double-stranded or single-stranded (i.e., a sense or an antisense single strand). The term transgenic is used broadly herein and refers to a genetically modified organism or genetically engineered organism whose genetic material has been altered using genetic engineering techniques. A knockout artiodactyl is thus transgenic regardless of whether or not exogenous genes or nucleic acids are expressed in the animal or its progeny.

### “Genetically Modified” AND “Genome Edited” Animals

Animals may be modified using various genetic engineering tools, including recombinase fusion proteins, or various vectors that are known. A genetic modification made by such tools may comprise disruption of a gene. Specific genome editing can be accomplished with targeting endonucleases such as TALENs, CRISPR/Cas9, ZFNs, meganucleases other nucleases and methods of specifically changing the base residues of a cells native genomic complement. As such, gene editing or genome editing does not add foreign DNA into a host's cell in contrast to transgenic methods. The term disruption of a gene refers to preventing the formation of a functional gene product. A gene product is functional only if it fulfills its normal (wild-type) functions. Disruption of the gene prevents expression of a functional factor encoded by the gene and comprises an insertion, deletion, or substitution of one or more bases in a sequence encoded by the gene and/or a promoter and/or an operator that is necessary for expression of the gene in the animal. The disrupted gene may be disrupted by, e.g., removal of at least a portion of the gene from a genome of the animal, alteration of the gene to prevent expression of a functional factor encoded by the gene, an interfering RNA, or expression of a dominant negative factor by an exogenous gene. Materials and methods of genetically modifying and/or genome editing animals are further detailed in U.S. Pat. No. 8,518,701; U.S. 2010/0251395; and U.S. 2012/0222143 which are hereby incorporated by reference for all purposes; in case of conflict, the instant specification is controlling. The term trans-acting refers to processes acting on a target gene from a different molecule (i.e., intermolecular). A trans-acting element is usually a DNA sequence that contains a gene. This gene codes for a protein (or microRNA or other diffusible molecule) that is used in the regulation the target gene. The trans-acting gene may be on the same chromosome as the target gene, but the activity is via the intermediary protein or RNA that it encodes. Embodiments of trans-acting gene are, e.g., genes that encode targeting endonucleases. Inactivation of a gene using a dominant negative generally involves a trans-acting element. The term cis-regulatory or cis-acting means an action without coding for protein or RNA; in the context of gene inactivation, this generally means inactivation of the coding portion of a gene, or a promoter and/or operator that is necessary for expression of the functional gene.

Various techniques known in the art can be used to inactivate genes to make knock-out animals and/or to introduce nucleic acid constructs into animals to produce founder animals and to make animal lines, in which the knockout or nucleic acid construct is integrated into the genome. Such techniques include, without limitation, pronuclear microinjection (U.S. Pat. No. 4,873,191), retrovirus mediated gene transfer into germ lines (Van der Putten et al., Proc. Natl. Acad. Sci. USA, 82:6148-6152, 1985), gene targeting into embryonic stem cells (Thompson et al., Cell, 56:313-321, 1989), electroporation of embryos (Lo, Mol. Cell. Biol., 3:1803-1814, 1983), sperm-mediated gene transfer (Lavitrano et al., Proc. Natl. Acad. Sci. USA, 99:14230-14235, 2002; Lavitrano et al., Reprod. Fert. Develop., 18:19-23, 2006), and in vitro transformation of somatic cells, such as cumulus or mammary cells, or adult, fetal, or embryonic stem cells, followed by nuclear transplantation (Wilmut et al., Nature, 385:810-813, 1997; and Wakayama et al., Nature, 394:369-374, 1998). Pronuclear microinjection, sperm mediated gene transfer, and somatic cell nuclear transfer are particularly useful techniques. An animal that is genomically modified is an animal wherein all of its cells have the genetic modification, including its germ line cells. When methods are used that produce an animal that is mosaic in its genetic modification, the animals may be inbred and progeny that are genomically modified may be selected. Cloning, for instance, may be used to make a mosaic animal if its cells are modified at the blastocyst state, or genomic modification can take place when a single-cell is modified. Animals that are modified so they do not sexually mature can be homozygous or heterozygous for the modification, depending on the specific approach that is used. If a particular gene is inactivated by a knock out modification, homozygosity would normally be required. If a particular gene is inactivated by an RNA interference or dominant negative strategy, then heterozygosity is often adequate.

Typically, in pronuclear microinjection, a nucleic acid construct is introduced into a fertilized egg; 1 or 2 cell fertilized eggs are used as the pronuclei containing the genetic material from the sperm head and the egg are visible within the protoplasm. Pronuclear staged fertilized eggs can be obtained in vitro or in vivo (i.e., surgically recovered from the oviduct of donor animals). In vitro fertilized eggs can be produced as follows. For example, swine ovaries can be collected at an abattoir, and maintained at 22-28° C. during transport. Ovaries can be washed and isolated for follicular aspiration, and follicles ranging from 4-8 mm can be aspirated into 50 mL conical centrifuge tubes using 18-gauge needles and under vacuum. Follicular fluid and aspirated oocytes can be rinsed through pre-filters with commercial TL-HEPES (Minitube, Verona, Wis.). Oocytes surrounded by a compact cumulus mass can be selected and placed into TCM-199 OOCYTE MATURATION MEDIUM (Minitube, Verona, Wis.) supplemented with 0.1 mg/mL cysteine, 10 ng/mL epidermal growth factor, 10% porcine follicular fluid, 50 μM 2-mercaptoethanol, 0.5 mg/ml cAMP, 10 IU/mL each of pregnant mare serum gonadotropin (PMSG) and human chorionic gonadotropin (hCG) for approximately 22 hours in humidified air at 38.7° C. and 5% CO₂. Subsequently, the oocytes can be moved to fresh TCM-199 maturation medium, which will not contain cAMP, PMSG or hCG and incubated for an additional 22 hours. Matured oocytes can be stripped of their cumulus cells by vortexing in 0.1% hyaluronidase for 1 minute.

For swine, mature oocytes can be fertilized in 500 μl Minitube PORCPRO IVF MEDIUM SYSTEM (Minitube, Verona, Wis.) in Minitube 5-well fertilization dishes. In preparation for in vitro fertilization (IVF), freshly-collected or frozen boar semen can be washed and resuspended in PORCPRO IVF Medium to 4×10⁵ sperm. Sperm concentrations can be analyzed by computer assisted semen analysis (SPERMVISION, Minitube, Verona, Wis.). Final in vitro insemination can be performed in a 10 μl volume at a final concentration of approximately 40 motile sperm/oocyte, depending on boar. Incubate all fertilizing oocytes at 38.7° C. in 5.0% CO₂ atmosphere for 6 hours. Six hours post-insemination, presumptive zygotes can be washed twice in NCSU-23 and moved to 0.5 mL of the same medium. This system can produce 20-30% blastocysts routinely across most boars with a 10-30% polyspermic insemination rate.

Linearized nucleic acid constructs can be injected into one of the pronuclei. Then the injected eggs can be transferred to a recipient female (e.g., into the oviducts of a recipient female) and allowed to develop in the recipient female to produce the transgenic animals. In particular, in vitro fertilized embryos can be centrifuged at 15,000×g for 5 minutes to sediment lipids allowing visualization of the pronucleus. The embryos can be injected with an Eppendorf FEMTOJET injector and can be cultured until blastocyst formation. Rates of embryo cleavage and blastocyst formation and quality can be recorded.

Embryos can be surgically transferred into uteri of asynchronous recipients. Typically, 100-200 (e.g., 150-200) embryos can be deposited into the ampulla-isthmus junction of the oviduct using a 5.5-inch TOMCAT® catheter. After surgery, real-time ultrasound examination of pregnancy can be performed.

In somatic cell nuclear transfer, a transgenic artiodactyl cell (e.g., a transgenic pig cell or bovine cell) such as an embryonic blastomere, fetal fibroblast, adult ear fibroblast, or granulosa cell that includes a nucleic acid construct described above, can be introduced into an enucleated oocyte to establish a combined cell. Oocytes can be enucleated by partial zona dissection near the polar body and then pressing out cytoplasm at the dissection area. Typically, an injection pipette with a sharp beveled tip is used to inject the transgenic cell into an enucleated oocyte arrested at meiosis 2. In some conventions, oocytes arrested at meiosis-2 are termed eggs. After producing a porcine or bovine embryo (e.g., by fusing and activating the oocyte), the embryo is transferred to the oviducts of a recipient female, about 20 to 24 hours after activation. See, for example, Cibelli et al., Science, 280:1256-1258, 1998; and U.S. Pat. No. 6,548,741. For pigs, recipient females can be checked for pregnancy approximately 20-21 days after transfer of the embryos.

Standard breeding techniques can be used to create animals that are homozygous for the exogenous nucleic acid from the initial heterozygous founder animals. Homozygosity may not be required, however. Transgenic pigs described herein can be bred with other pigs of interest.

In some embodiments, a nucleic acid of interest and a selectable marker can be provided on separate transposons and provided to either embryos or cells in unequal amount, where the amount of transposon containing the selectable marker far exceeds (5-10-fold excess) the transposon containing the nucleic acid of interest. Transgenic cells or animals expressing the nucleic acid of interest can be isolated based on presence and expression of the selectable marker. Because the transposons will integrate into the genome in a precise and unlinked way (independent transposition events), the nucleic acid of interest and the selectable marker are not genetically linked and can easily be separated by genetic segregation through standard breeding. Thus, transgenic animals can be produced that are not constrained to retain selectable markers in subsequent generations, an issue of some concern from a public safety perspective.

Once transgenic animals have been generated, expression of an exogenous nucleic acid can be assessed using standard techniques. Initial screening can be accomplished by Southern blot analysis to determine whether or not integration of the construct has taken place. For a description of Southern analysis, see sections 9.37-9.52 of Sambrook et al., Molecular Cloning, A Laboratory Manual, second edition, Cold Spring Harbor Press, Plainview; N.Y., 1989. Polymerase chain reaction (PCR) techniques also can be used in the initial screening. PCR refers to a procedure or technique in which target nucleic acids are amplified. Generally, sequence information from the ends of the region of interest or beyond is employed to design oligonucleotide primers that are identical or similar in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. PCR is described in, for example PCR Primer: A Laboratory Manual, ed. Dieffenbach and Dveksler, Cold Spring Harbor Laboratory Press, 1995. Nucleic acids also can be amplified by ligase chain reaction, strand displacement amplification, self-sustained sequence replication, or nucleic acid sequence-based amplified. See, for example, Lewis, Genetic Engineering News, 12:1, 1992; Guatelli et al., Proc. Natl. Acad. Sci. USA, 87:1874, 1990; and Weiss, Science, 254:1292, 1991. At the blastocyst stage, embryos can be individually processed for analysis by PCR, Southern hybridization and splinkerette PCR (see, e.g., Dupuy et al., Proc Natl Acad Sci USA, 99:4495, 2002).

Expression of a nucleic acid sequence encoding a polypeptide in the tissues of transgenic pigs can be assessed using techniques that include, for example, Northern blot analysis of tissue samples obtained from the animal, in situ hybridization analysis, Western analysis, immunoassays such as enzyme-linked immunosorbent assays, and reverse-transcriptase PCR (RT-PCR).

### Interfering RNAs

A variety of interfering RNA (RNAi) are known. Double-stranded RNA (dsRNA) induces sequence-specific degradation of homologous gene transcripts. RNA-induced silencing complex (RISC) metabolizes dsRNA to small 21-23-nucleotide small interfering RNAs (siRNAs). RISC contains a double stranded RNAse (dsRNase, e.g., Dicer) and ssRNase (e.g., Argonaut 2 or Ago2). RISC utilizes antisense strand as a guide to find a cleavable target. Both siRNAs and microRNAs (miRNAs) are known. A method of disrupting a gene in a genetically modified animal comprises inducing RNA interference against a target gene and/or nucleic acid such that expression of the target gene and/or nucleic acid is reduced.

For example, the exogenous nucleic acid sequence can induce RNA interference against a nucleic acid encoding a polypeptide. For example, double-stranded small interfering RNA (siRNA) or small hairpin RNA (shRNA) homologous to a target DNA can be used to reduce expression of that DNA. Constructs for siRNA can be produced as described, for example, in Fire et al., Nature, 391:806, 1998; Romano and Masino, Mol. Microbiol., 6:3343, 1992; Cogoni et al., EMBO J., 15:3153, 1996; Cogoni and Masino, Nature, 399:166, 1999; Misquitta and Paterson Proc. Natl. Acad. Sci. USA, 96:1451, 1999; and Kennerdell and Carthew, Cell, 95:1017, 1998. Constructs for shRNA can be produced as described by McIntyre and Fanning (2006) BMC Biotechnology 6:1. In general, shRNAs are transcribed as a single-stranded RNA molecule containing complementary regions, which can anneal and form short hairpins.

The probability of finding a single, individual functional siRNA or miRNA directed to a specific gene is high. The predictability of a specific sequence of siRNA, for instance, is about 50% but a number of interfering RNAs may be made with good confidence that at least one of them will be effective.

Embodiments include an in vitro cell, an in vivo cell, and a genetically modified animal such as a livestock animal that express an RNAi directed against a gene, e.g., a gene selective for a developmental stage. The RNAi may be, for instance, selected from the group consisting of siRNA, shRNA, dsRNA, RISC and miRNA.

### Inducible Systems

An inducible system may be used to control expression of a gene. Various inducible systems are known that allow spatiotemporal control of expression of a gene. Several have been proven to be functional in vivo in transgenic animals. The term inducible system includes traditional promoters and inducible gene expression elements.

An example of an inducible system is the tetracycline (tet)-on promoter system, which can be used to regulate transcription of the nucleic acid. In this system, a mutated Tet repressor (TetR) is fused to the activation domain of herpes simplex virus VP16 trans-activator protein to create a tetracycline-controlled transcriptional activator (tTA), which is regulated by tet or doxycycline (dox). In the absence of antibiotic, transcription is minimal, while in the presence of tet or dox, transcription is induced. Alternative inducible systems include the ecdysone or rapamycin systems. Ecdysone is an insect molting hormone whose production is controlled by a heterodimer of the ecdysone receptor and the product of the ultraspiracle gene (USP). Expression is induced by treatment with ecdysone or an analog of ecdysone such as muristerone A. The agent that is administered to the animal to trigger the inducible system is referred to as an induction agent.

The tetracycline-inducible system and the Cre/loxP recombinase system (either constitutive or inducible) are among the more commonly used inducible systems. The tetracycline-inducible system involves a tetracycline-controlled transactivator (tTA)/reverse tTA (rtTA). A method to use these systems in vivo involves generating two lines of genetically modified animals. One animal line expresses the activator (tTA, rtTA, or Cre recombinase) under the control of a selected promoter. Another set of transgenic animals express the acceptor, in which the expression of the gene of interest (or the gene to be modified) is under the control of the target sequence for the tTA/rtTA transactivators (or is flanked by loxP sequences). Mating the two strains of mice provides control of gene expression.

The tetracycline-dependent regulatory systems (tet systems) rely on two components, i.e., a tetracycline-controlled transactivator (tTA or rtTA) and a tTA/rtTA-dependent promoter that controls expression of a downstream cDNA, in a tetracycline-dependent manner. In the absence of tetracycline or its derivatives (such as doxycycline), tTA binds to tetO sequences, allowing transcriptional activation of the tTA-dependent promoter. However, in the presence of doxycycline, tTA cannot interact with its target and transcription does not occur. The tet system that uses tTA is termed tet-OFF because tetracycline or doxycycline allows transcriptional down-regulation. Administration of tetracycline or its derivatives allows temporal control of transgene expression in vivo. rtTA is a variant of tTA that is not functional in the absence of doxycycline but requires the presence of the ligand for transactivation. This tet system is therefore termed tet-ON. The tet systems have been used in vivo for the inducible expression of several transgenes, encoding, e.g., reporter genes, oncogenes, or proteins involved in a signaling cascade.

The Cre/lox system uses the Cre recombinase, which catalyzes site-specific recombination by crossover between two distant Cre recognition sequences, i.e., loxP sites. A DNA sequence introduced between the two loxP sequences (termed floxed DNA) is excised by Cre-mediated recombination. Control of Cre expression in a transgenic animal, using either spatial control (with a tissue- or cell-specific promoter) or temporal control (with an inducible system), results in control of DNA excision between the two loxP sites. One application is for conditional gene inactivation (conditional knockout). Another approach is for protein over-expression, wherein a floxed stop codon is inserted between the promoter sequence and the DNA of interest. Genetically modified animals do not express the transgene until Cre is expressed, leading to excision of the floxed stop codon. This system has been applied to tissue-specific oncogenesis and controlled antigen receptor expression in B lymphocytes. Inducible Cre recombinases have also been developed. The inducible Cre recombinase is activated only by administration of an exogenous ligand. The inducible Cre recombinases are fusion proteins containing the original Cre recombinase and a specific ligand-binding domain. The functional activity of the Cre recombinase is dependent on an external ligand that is able to bind to this specific domain in the fusion protein. More recently an engineered Cre recombinase has been designed, CreERT2. CreERT2 encodes a Cre recombinase (Cre) fused to a mutant estrogen ligand-binding domain (ERT2) that requires the presence of tamoxifen for activity.

In some embodiments, the inducible system is temporally and/or tissue specific. For example, the Cre enzyme can be expressed as a fusion protein with a mutant estrogen receptor ligand-binding domain which is exclusively responsive to the synthetic estrogen receptor antagonist, Tamoxifen (Schwenk et al. 1998). Other embodiments include use of tissue specific promoters. For example, promoters of genes that are only expressed in specific tissue can be used to drive transgenes in desired tissues. For instance, some genes, when disrupted, selectively interfere with spermatogenesis and prevent, or destroy, formation of a gamete. Genes in the DAZ family, DAZL, and DAZ1. DAZ1 is selective for gametogenesis, specifically, spermatogenesis, with disruption causing no sperm to form. DAZ1 is on the Y-chromosome. Other genes important in gametogenesis include NANOS3 and VASA.

The founder DAZL−/− boars were developed using TALEN stimulated homology dependent repair and followed by cloning. Outside of some minor flexor tendon abnormalities common to cloning, there was no visible phenotype in the founders and they displayed typical boar behavior; aggressiveness, strong odor, mounting, at the onset of puberty. Once they reached 7 month of age, the boars were trained for semen collection. In a blind evaluation, microscopic analysis of 3-serial ejaculates collected from the DAZL−/− boars showed no detectable sperm demonstrating achievement of Milestone 1. These findings were confirmed in ejaculates concentrated by centrifugation (data not shown). Milestone 2. Characterize spermatogenesis in DAZL−/− testes.

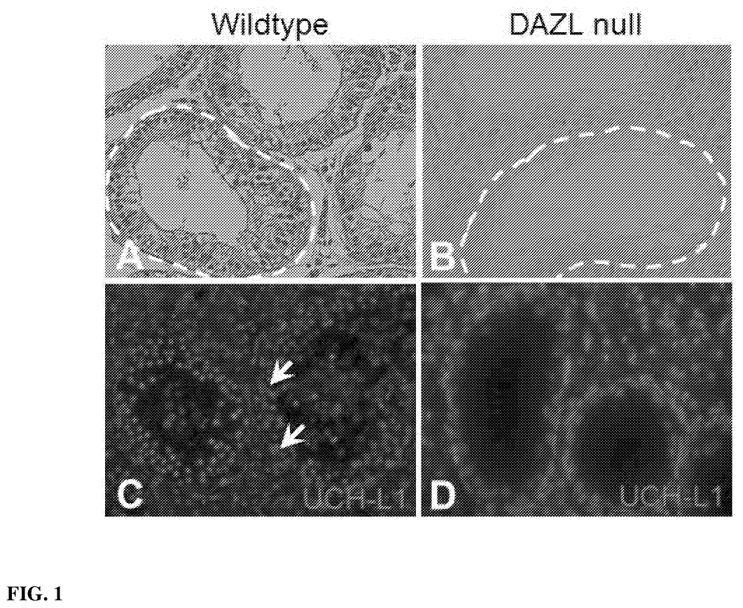

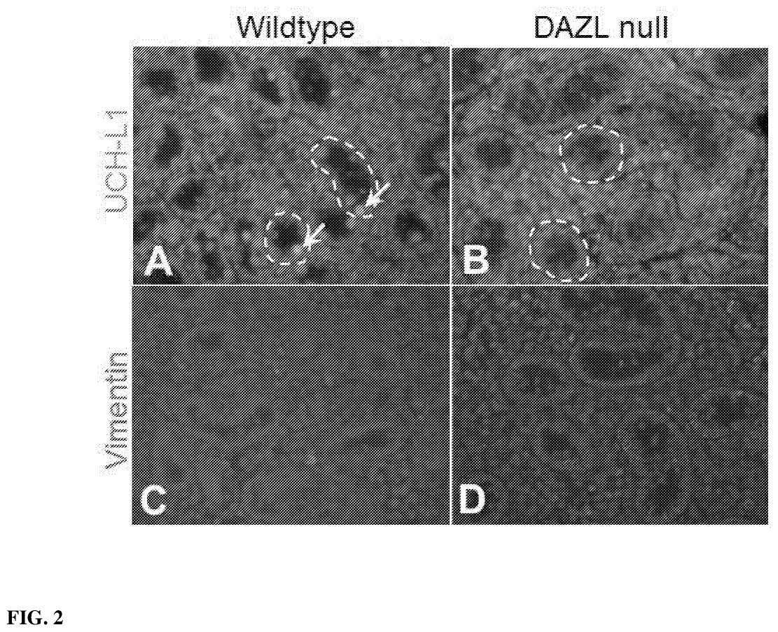

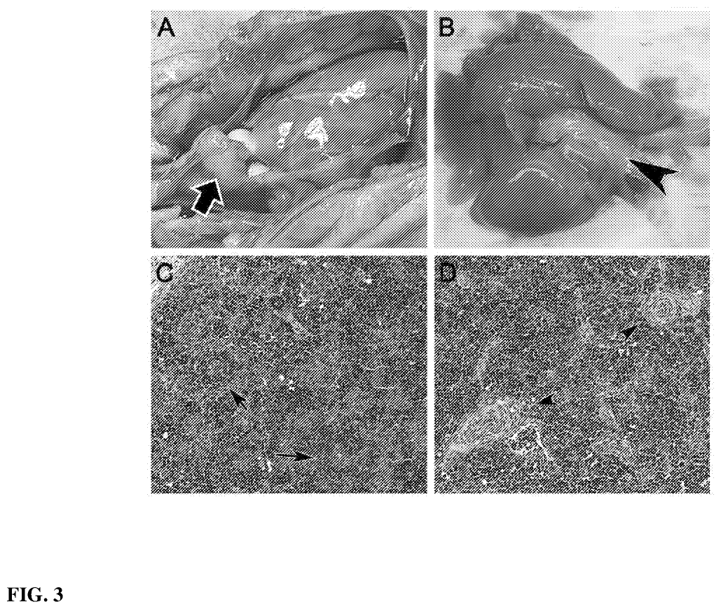

Histological evaluation of cross sections of adult DAZL−/− testes revealed intact seminiferous tubules completely devoid of germ cells within the lumen suggesting spermatogenic failure (FIG. 1). To further characterize the DAZL−/− spermatogenic failure phenotype, cross sections from 10 week and adult DAZL−/− testes were analyzed for expression of germ cell and somatic cell markers by immunohistochemistry (FIG. 2). Consistent with the absence of germ cells in seminiferous tubules in hematoxylin and eosin stained sections, no expression of type A spermatogonia cell marker UCH-L134 was observed in adult (FIG. 3) or 10-week-old testes sections. Taken together, this indicates that the failure of spermatogenesis in the DAZL−/− boars is due to the absence of germline stem cells. In Dazl knockout mice, the loss of spermatogenesis coincides with a 3.4-fold reduction in testis mass compared to wildtype48. Surprisingly, in DAZL−/− porcine testis a reduction in mass was not observed.

Within the seminiferous tubules, somatic Sertoli cells provide structural and functional support to germ cells and are required for spermatogenesis49. To examine the effect of DAZL−/− on Sertoli cell morphology 10 wk old DAZL−/− and WT testes sections were labeled with vimentin, an intermediate filament marker and indicator of the structural integrity of the seminiferous epithelium50. The loss of vimentin expression is associated with spermatogenic dysfunction. Vimentin expression in DAZL−/− testes was similar to that observed in WT testes confirming that although germ cells are absent in the DAZL−/− testes, the seminiferous tubule morphology remains intact. The absence of germ cells by 10 weeks of age in the DAZL−/− testes and the preservation of tubule morphology suggest that the DAZL−/− testes is an ideal environment for GST or blastocyst complementation.

Other embodiments include an in vitro cell, an in vivo cell, and a genetically modified or genome edited animal such as a livestock animal that comprise a gene under control of an inducible system. The genetic modification of an animal may be genomic or mosaic. The inducible system may be, for instance, selected from the group consisting of Tet-On, Tet-Off, Cre-lox, and Hif1 alpha. An embodiment is a gene set forth herein.

### Dominant Negatives

Genes may thus be disrupted not only by removal or RNAi suppression but also by creation/expression of a dominant negative variant of a protein which has inhibitory effects on the normal function of that gene product. The expression of a dominant negative (DN) gene can result in an altered phenotype, exerted by a) a titration effect; the DN PASSIVELY competes with an endogenous gene product for either a cooperative factor or the normal target of the endogenous gene without elaborating the same activity, b) a poison pill (or monkey wrench) effect wherein the dominant negative gene product ACTIVELY interferes with a process required for normal gene function, c) a feedback effect, wherein the DN ACTIVELY stimulates a negative regulator of the gene function.

### Founder Animals, Animal Lines, Traits, and Reproduction

Founder animals (F₀ generation) may be produced by cloning and other methods described herein. The founders can be homozygous for a genetic modification or genome edit, as in the case where a zygote or a primary cell undergoes a homozygous modification. Similarly, founders can also be made that are heterozygous. The founders may be genomically modified or gene edited, meaning that the cells in their genome have undergone modification or edits. Founders can be mosaic for a modification or edit, as may happen when vectors are introduced into one of a plurality of cells in an embryo, typically at a blastocyst stage. Progeny of mosaic animals may be tested to identify progeny that are genomically modified or edited. An animal line is established when a pool of animals has been created that can be reproduced sexually or by assisted reproductive techniques, with heterogeneous or homozygous progeny consistently expressing the modification or edit.

In livestock, many alleles are known to be linked to various traits such as production traits, type traits, workability traits, and other functional traits. Artisans are accustomed to monitoring and quantifying these traits, e.g., Visscher et al., Livestock Production Science, 40:123-137, 1994; U.S. Pat. No. 7,709,206; U.S. 2001/0016315; U.S. 2011/0023140; and U.S. 2005/0153317. An animal line may include a trait chosen from a trait in the group consisting of a production trait, a type trait, a workability trait, a fertility trait, a mothering trait, and a disease resistance trait. Further traits include expression of a recombinant gene product.

### Recombinases

Embodiments of the invention include administration of a targeted nuclease system with a recombinase (e.g., a RecA protein, a Rad51) or other DNA-binding protein associated with DNA recombination. A recombinase forms a filament with a nucleic acid fragment and, in effect, searches cellular DNA to find a DNA sequence substantially homologous to the sequence. For instance, a recombinase may be combined with a nucleic acid sequence that serves as a template for HDR. The recombinase is then combined with the HDR template to form a filament and placed into the cell. The recombinase and/or HDR template that combines with the recombinase may be placed in the cell or embryo as a protein, an mRNA, or with a vector that encodes the recombinase. The disclosure of U.S. 2011/0059160 (U.S. patent application Ser. No. 12/869,232) is hereby incorporated herein by reference for all purposes; in case of conflict, the specification is controlling. The term recombinase refers to a genetic recombination enzyme that enzymatically catalyzes, in a cell, the joining of relatively short pieces of DNA between two relatively longer DNA strands. Recombinases include Cre recombinase, Hin recombinase, RecA, RAD51, Cre, and FLP. Cre recombinase is a Type I topoisomerase from P1 bacteriophage that catalyzes site-specific recombination of DNA between loxP sites. Hin recombinase is a 21 kD protein composed of 198 amino acids that is found in the bacteria Salmonella. Hin belongs to the serine recombinase family of DNA invertases in which it relies on the active site serine to initiate DNA cleavage and recombination. RAD51 is a human gene. The protein encoded by this gene is a member of the RAD51 protein family which assists in repair of DNA double strand breaks. RAD51 family members are homologous to the bacterial RecA and yeast Rad51. Cre recombinase is an enzyme that is used in experiments to delete specific sequences that are flanked by loxP sites. FLP refers to Flippase recombination enzyme (FLP or Flp) derived from the 2μ plasmid of the baker's yeast Saccharomyces cerevisiae.

In some embodiments, a sequence encoding a selectable marker can be flanked by recognition sequences for a recombinase such as, e.g., Cre or Flp. For example, the selectable marker can be flanked by loxP recognition sites (34-bp recognition sites recognized by the Cre recombinase) or FRT recognition sites such that the selectable marker can be excised from the construct. See, Orban, et al., Proc. Natl. Acad. Sci. 89:6861, 1992, for a review of Cre/lox technology, and Brand and Dymecki, Dev. Cell 6:7, 2004. A transposon containing a Cre- or Flp-activatable transgene interrupted by a selectable marker gene also can be used to obtain transgenic animals with conditional expression of a transgene. For example, a promoter driving expression of the marker/transgene can be either ubiquitous or tissue-specific, which would result in the ubiquitous or tissue-specific expression of the marker in F0 animals (e.g., pigs). Tissue specific activation of the transgene can be accomplished, for example, by crossing a pig that ubiquitously expresses a marker-interrupted transgene to a pig expressing Cre or Flp in a tissue-specific manner, or by crossing a pig that expresses a marker-interrupted transgene in a tissue-specific manner to a pig that ubiquitously expresses Cre or Flp recombinase. Controlled expression of the transgene or controlled excision of the marker allows expression of the transgene.

Herein, “RecA” or “RecA protein” refers to a family of RecA-like recombination proteins having essentially all or most of the same functions, particularly: (i) the ability to position properly oligonucleotides or polynucleotides on their homologous targets for subsequent extension by DNA polymerases; (ii) the ability topologically to prepare duplex nucleic acid for DNA synthesis; and, (iii) the ability of RecA/oligonucleotide or RecA/polynucleotide complexes efficiently to find and bind to complementary sequences. The best characterized RecA protein is from E. coli; in addition to the original allelic form of the protein a number of mutant RecA-like proteins have been identified, for example, RecA803. Further, many organisms have RecA-like strand-transfer proteins including, for example, yeast, Drosophila, mammals including humans, and plants. These proteins include, for example, Rec1, Rec2, Rad51, Rad51B, Rad51C, Rad51D, Rad51E, XRCC2 and DMC1. An embodiment of the recombination protein is the RecA protein of E. coli. Alternatively, the RecA protein can be the mutant RecA-803 protein of E. coli, a RecA protein from another bacterial source or a homologous recombination protein from another organism.

Precise Integration into Target Chromosome (PITCh)

PITCh as used herein refers to Precise Integration into Target Chromosome. PITCh is a gene knock-in approach based on microhomology-mediated end-joining and or SSA—the exact mechanism not yet determined. In the PITCh system, the targeting vector and the genomic target site are simultaneously cut by TALENs or CRISPR (TAL-PITCh or CRISP-PITCh respectively), then the linearized DNA fragment is integrated into the genome via short microhomologies in the range of 8-72 bp. In some instances, generic single-guide RNA (sgRNA) are used to cleave the PITCh donor vector.

In double stranded PITCh a template, contained within a plasmid, is introduced into the cell at about the same time as a nuclease. The template is liberated from the plasmid by the introduction of an appropriate restriction enzyme at about the same time. In some embodiments, the insert is liberated from the plasmid by cas9 endonuclease. While the exact mechanism by which HDR is introduced into a genome by the cell is unknown, the inventors' experiments show that double stranded DNA when provided at about the same time as a targeted double stranded break (DSB) is made requires less template and requires much shorter homology arms than an ssODN template. In these examples, those of skill in the art will appreciate that that is a range of ratios of nuclease to plasmid to enzyme that can be empirically validated to achieve optimum HDR and that, in some instances, the ratio is determined by the size of the template that is used to make the deletion or insertion edited into the genome.

### Homology-Independent Targeted Integration (HITI)

HITI allows insertion of transgenes into both proliferating and non-proliferating cells. HITI targets an insertion site using CRISPR/Cas9, supplies an excess of linear DNA template, and allows the cells to insert the DNA template between the ends of the cut target DNA via NHEJ. If the cell anneals the two ends back together without the insert (or a mutation), the Cas9 target site would re-form and get cut again. Similarly, the designed donor DNA can be designed so that it also re-forms the cut site if it goes in backwards, ensuring that most insertions are the correct orientation. In addition, continued cleavage by Cas9 results in gRNA that is no longer able to bind to target sequences due to errors during NHEJ repair

### Compositions and Kits

The present invention also provides compositions and kits containing, for example, nucleic acid molecules encoding site-specific endonucleases, CRISPR, Cas9, ZNFs, TALENs, RecA-gal4 fusions, polypeptides of the same, compositions containing such nucleic acid molecules or polypeptides, or engineered cell lines. An HDR may also be provided that is effective for introgression of an indicated allele. Such items can be used, for example, as research tools, or therapeutically.

### Genetic Complementation

Classically, genetic complementation, refers to the production of a wild-type phenotype when two different mutations are combined in a diploid or a heterokaryon. However, modern techniques of chimera production can now rely on stem cell complementation, whereby cells of more than one embryonic origin are combined to make one genetically mixed animal. In this case, complementation does not involve any change in the genotypes of individual chromosomes; rather it represents the mixing of gene products. Complementation occurs during the time that two cell types are in the same embryo and can each supply a function. Afterward, each respective chromosome remains unaltered. In the case of chimeras, complementation occurs when two different sets of chromosomes, are active in the same embryo. However, progeny that result from this complementation can carry cells of each genotype. In embryonic complementation, genes of the host embryo are edited to produce a knock out or otherwise make a non-functional gene. When human stem cells are injected into the gene edited blastocyst, they can rescue or “complement” the defects of the host (edited) genome. When the gene or genes that are knocked out support the growth of a particular organ or tissue, the resulting complementation produced tissue can be the result of the growth and differentiation of the non-edited, e.g., stem cell derived genotype. When human stem cells are used to complement the host-edited genome, the resulting tissue or organ can be composed of human cells. In this way, fully human organs can be produced, in vivo, using an animal as a host for the complementation produced organ.

Because multiple genes may be responsible for the growth and differentiation of any particular organ or tissue, processes for multiplex gene edits are also described. See, for example, WO2015/027995, hereby incorporated by reference in its entirety. Multiple genes can be modified or knocked out in a cell or embryo that may be used for research or to make whole chimeric animals. These embodiments include the complementation of cell or organ loss by selective depopulation of host niches. See, for example, WO2017/075276, hereby incorporated by reference in its entirety. These inventions provide for rapid creation of animals to serve as models, food, and as sources of cellular and a cellular products for industry and medicine.

In regenerative medicine, swine can provide particular benefits with two primary goals. 1) To develop better large animal models of human disease for preclinical testing by gene editing. All novel therapies in regenerative medicine, pharmaceuticals, and medical devices are required to demonstrate safety and efficacy in animal models prior to entering human trials. Heavy reliance on rodent preclinical models has resulted in inflated failure rates due to vast differences in size, anatomy and physiology compared to humans. Pigs are widely considered the best large animal model of humans, and our goal is to develop lines of pigs that precisely mimic the human disease state leading to more relevant preclinical testing and reduced risk/cost associated with human clinical trials. 2). Engineer in vivo niches into swine to enable manufacturing of personalized human cells, tissues, and organs for research or transplantation. Immunodeficient swine serve both of these objectives in a variety of ways. First, an immunodeficient pig will allow direct assessment of human cell-based therapies in a large animal that will not reject the graft. In combination with other gene-edited lines of human disease, congenital heart failure as an example, would allow our colleagues to conduct safety and efficacy testing in the large animal model with human stem cells prepared using the established clinical protocol¹. Together with additional mutations, in vivo niches for regeneration in other cell types can be created. For example, it has been hypothesized that immunodeficient pigs with fumarylacetoacetate hydrolase (FAH) knockout may permit expansion of human hepatocytes in swine². Finally, establishment of an in vivo niche in the immunodeficient swine not only creates a platform to propagate human lymphocytes, but could also be an important step towards humanization of the swine immune system. Swine with a humanized immune system could have value for studying graft rejection and preclinical evaluation of biologic pharmaceuticals. As with immunodeficient rodents, swine with immunodeficiency have broad applications. However, unlike rodents, propagation of immunodeficient swine is a significant and costly challenge. the development of inducible immunodeficient swine will solve this problem and drive innovation in the industry.

Gene knockouts in blastocysts can create a niche in which normal syngeneic or xenogeneic stem cells should occupy to contribute to the development of the desired organ or cell (FIG. 1). Novel gene editing and gene modulation technologies using TALENS, REGENs such as CRISPR, and synthetic porcine artificial chromosomes are used to knockout desired target genes and to enhance the function of other genes that can minimize off-target effects.

Continued innovation in human cell based regenerative medicine has led to a dramatic increase in the number of new cell-based investigational drugs (INDs) submitted to the FDA. Between 2006 and 2013, 163 INDs involving cell-based therapies were filed with a range of clinical indications, the largest proportions of which related to cardiovascular therapy (27% of INDs)³. The number of new submissions is rising and is expected to continue into the foreseeable future. These cell-based therapies are very heterogeneous with differences in the source of the therapeutic cells, isolation and treatments of the cells, dosage and delivery of the cells. Therefore, cellular therapy has to pass through several levels of preclinical testing to justify human clinical trials. Preclinical evaluations should ideally 1) establish the scientific rationale for the therapeutics, 2) investigate the route of administration and characterize local and systemic toxicities of the therapeutic agent, 3) carry out dosage escalation studies to determine the dosing range and a safe starting dose for clinical trials and 4) determine which groups of patients to the therapeutic regimen could benefit and establish a clinical monitoring scheme. Choosing the correct animal model for preclinical testing is critical to generate the most relevant results.

Accordingly, the inventors have developed a suite of genome edited swine to mimic a variety of human disease states, particularly those with the most significant health consequences including: cardiovascular, diabetes, cancer, and neurogenerative disorders. The ability to combine these models with immunodeficiency is very advantageous.

A second emphasis is to develop innovative solutions for the unmet need of human organs and tissues for preclinical testing, and ultimately, transplantation into patients. Our objective is to use the process of blastocyst complementation to grow human organs in a pig that has been genetically tailored to lack specific cells or organs. This process was first demonstrated in rodents where the pancreas of a donor rat was grown in a mouse lacking a pancreas⁴. The process was then replicated in pig where the pancreas of a donor pig was produced in a swine host engineered to lack a pancreas⁵. In these examples, both the mouse and the pig hosts were deficient for PDX1, the master regulator of pancreas development. Since the host was unable to produce a pancreas, the injected cells from a second (wild-type) source were able to fill the open niche and produce the desired tissue. As a critical next step, researchers in California have recently demonstrated that human cells can indeed survive in the developing porcine embryo and give rise to differentiated cell types⁶. The immunodeficient pig would make an ideal host for testing human cell engraftment into the immune system by blastocyst complementation. The inducible aspect would enable large scale production of high quality, in vivo produced host blastocysts. While exciting, the blastocysts stage is not the only time point suitable for engrafting human progenitor cells into a porcine host. In rodents, postnatal delivery is the primary time point of engraftment for human immune cells or hepatocytes^{7,8}. Also, in pigs, gene-corrected autologous hepatocytes have been infused postnatal to cure hereditary tyrosinemia type 1 due to FAH deficiency⁹. Hence; the immunodeficient pig will be a critical platform for postnatal delivery of human cells.

The impact of immunodeficient pigs is far-reaching. As with rodents, there are a number of applications for immunodeficient and humanized swine that extend beyond Regenerative Medicine. In cancer research and preclinical testing, applications include: 1) human-to-pig cancer xenograft models and drug testing; and 2) evaluation of the role of the immune system (humanized pig) in response to chemo- and radio-therapies for the treatment of cancer¹⁰. These animals may also have a major impact on immunological research and treatments including the evaluation of: 1) immune-modulatory drugs^{11,12}; 2) cell-based therapies¹³; 3) adoptive T-cell transfer¹⁴; 4) autologous immune enhancement therapy¹⁵; 5) genetically engineered T-cells¹⁶; and 6) studies of inflammation and infectious disease¹⁷. This incredible diversity of applications is far greater than any other genetically modified swine model that currently exists and it supports development of an innovative and sustainable solution to produce immunodeficient swine in a rapid and cost-effective manner.

### The Bottleneck:

Using targeting endonuclease (TALEN) mediated gene editing and SCNT, the inventors have developed RG-KO pigs and observed a lack of T, B, and NK cells (detailed below). To propagate these animals by conventional breeding, animals would need to be heterozygous for mutations in RAG2 and IL2Rg. With this breeding scheme, only ˜6% of offspring would have the desired phenotype in the F1 generation (Table 1). This is both cost prohibitive and technically challenging considering it would take nine litters to get a cohort of 3 RG-KO swine. Furthermore, as each litter is delivered by sterile c-section into a germ-free environment¹⁸, the logistics of RG-KO production by this scheme is untenable and would result in culling of 94% of the offspring. In contrast, breeding from the regRG-KO line between one or two regRG-KO parents would significantly increase the production rate of RG-KO offspring to 25 and 100 percent, respectively (Table 1).

TABLE 1
Breeding advantage of reg-RG-KO.

| | | | | Litters to |
|---|---|---|---|---|
| | | | | get 3 |
| Male | | Female | % RG-KO | RG-KOs^{a} |
| reg-RG-KO | X | Reg-RG-KO | 100%* | 1 |
| reg-RG-KO | X | IL2Rg^{+/-}; RAG2^{+/-} | 25%* | 2 |
| IL2Rg^{y/+}; RAG2^{+/-} | X | IL2Rg^{+/-}; RAG2^{+/-} | 6.3% | 9 |

^{a}Probability x > 0.90; considering litter size of 10.
*Assumes 100% efficacy of regRG-KO.

Accordingly, various exemplary embodiments of devices and compounds as generally described above and methods according to this invention, will be understood more readily by reference to the following examples, which are provided by way of illustration and are not intended to be limiting of the invention in any fashion.

### Example 1

### A Swine Model of X-Linked Severe Combined Immunodeficiency (XSCID)

In response to the need for immunodeficient swine, two groups have independently produced a swine model of x-linked severe combined immunodeficiency (XSCID) in pigs by knockout of the common gamma chain receptor component, IL2Rg^{19,20.} As anticipated, males with mutant alleles of IL2Rg were athymic and largely void of T and NK cells. Under standard housing conditions, XSCID piglets became systemically ill and could not be maintained to breeding age. In transplantation experiments conducted with these animals, researchers found that although XSCID pigs lacked T and NK cells, allogeneic engraftment rates were lower than expected, as bone marrow transplantation (BMT) restored immunity to only 3 of 5 individuals¹⁹. In addition, xenogeneic BMT with human cells was not successful and the authors speculated that disruption of additional factors, such as RAG1 or RAG2 would be required. RAG2-deficient mice lack the ability to undergo V(D)J recombination and therefor lack mature lymphocytes¹⁹. The recently established RAG2^{-/-} swine allowed engraftment of subcutaneous human iPS cell xenografts, albeit with variable success, presumably due to an NK cell population²¹. Considering the success of the FRG mouse (Fah^{-/-}; RAG2^{-/-}; IL2Rg^{-/-}) for engraftment of human hepatocytes²², we initiated a project to test the feasibility of knocking out both RAG2 and IL2Rg (RG-KO) by gene-editing, as opposed to breeding methods and thus producing the desired genotype in the F0 generation. Primary porcine fibroblasts with bi-allelic knockout of IL2Rg and RAG2 genes (“RG-KO”) were generated using the inventors standard GoldyTALEN platform²³. The resulting cell lines were used in somatic cell nuclear transfer to produce seven immunodeficient piglets and compared to eight new-born wild-type piglets was euthanized and served as comparison controls.

As expected, RG-KO piglets were devoid of thymuses (FIG. 3B). In addition, no peripheral or mesentery lymph nodes could be appreciated in these animals (data not shown). In comparison, the thymus was clearly observed in age-matched wild-type control piglets (FIG. 3A) while tissue samples were obtained that included numerous mesentery and peripheral lymph nodes (data not shown). Histological comparison analysis of hematoxylin/eosin (H&E) stained paraffin sections of the spleens of both sets of animals showed noticeable differences. RG-KO spleens were smaller than wild type and cells in RG-KOs were more loosely packed. Moreover, the periarterial lymphoid sheaths (PALS) that normally surround central arteries in the spleen were completely absent compared to wild-type animals (FIG. 3C, D). In addition, the presence of intraepithelial lymphocytes was absent in H&E stained sections of the intestine of immune deficient piglets (data not shown).

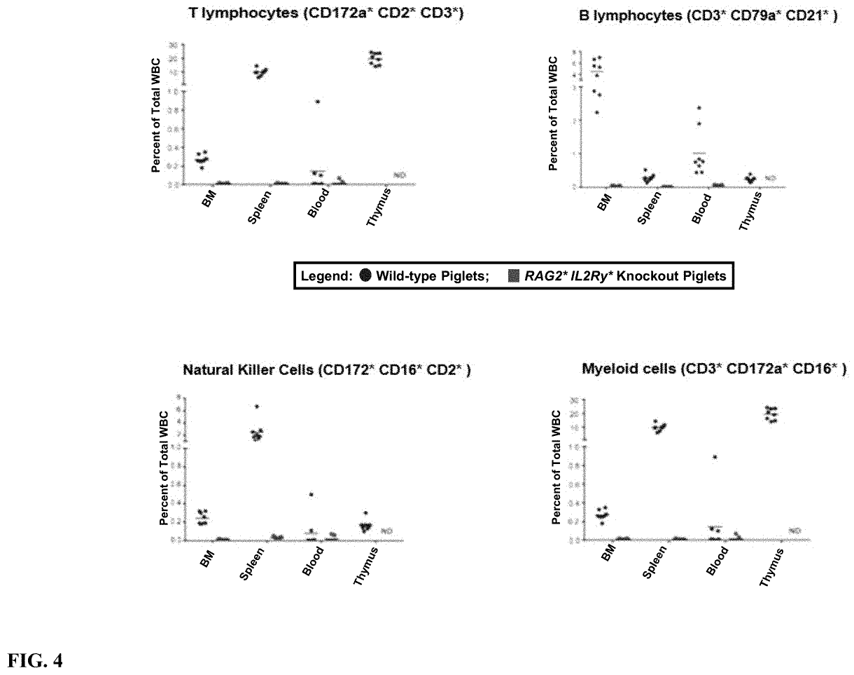

Flow cytometry analysis of cell populations isolated from blood and lymphoid organs of RG-KO piglets showed a complete absence of mature T cells, B cells and natural killer cells while populations of myeloid cells were equivalent to those of wild-type piglets (FIG. 4). Together, our RG-KO animals are shown to lack T, B and NK cells and represent a useful starting point for large-scale propagation with the inducible strategy proposed.

### Example 2

### Production of FAH/IL2Rg/Rag2/“FRG” Triple KO

The RG-KO pig was made using multiplex gene editing as reported in the inventors' prior application US PUB App. 2016/0029604 (U.S. Ser. No. 14/698,561) hereby incorporated in by reference in its entirety for all purposes. As noted above, the piglets lacked an immune system and were sacrificed in utero at 100 days of gestation. No structural abnormalities were noted in the RG-KO piglets. Accordingly, upon histologic analysis of the piglet's samples of primary cells (fibroblasts) were taken (ear punch) and preserved. In recognition of the importance of the further knockout of FAH together with IL2Rg and RAG2, TALENs were prepared to target FAH and an HDR oligo designed to introduce a unique HindIII restriction site as shown below.

FAH 5.3 TALEN pairs
5′ RVD:
NG HD NI NN NN NN NN HD NI NI NN NN NI NN NI HD NG
3′ RVD:
NN NN NG HD NG NN NN NN NI HD NI NG NI HD HD
(SEQ ID NO: 1)
5′ Binding site spacer 3′ binding site
TCAGGGGCAAGGAGACT gcactgatgcccaatt GGTATGTCCCAGACC
FAH 5.3 KO oligo
(SEQ ID NO: 2)
acaaacgtcggagtcatgttcaggggcaaggagactgcactgT [symbol]
cccaattggtatgtcccagaccagtgtctggctgagttct
Ital = Talen cut site
Arial Bold = stop codon
Underlined = HindIII restriction site
Upper case = inserted bases

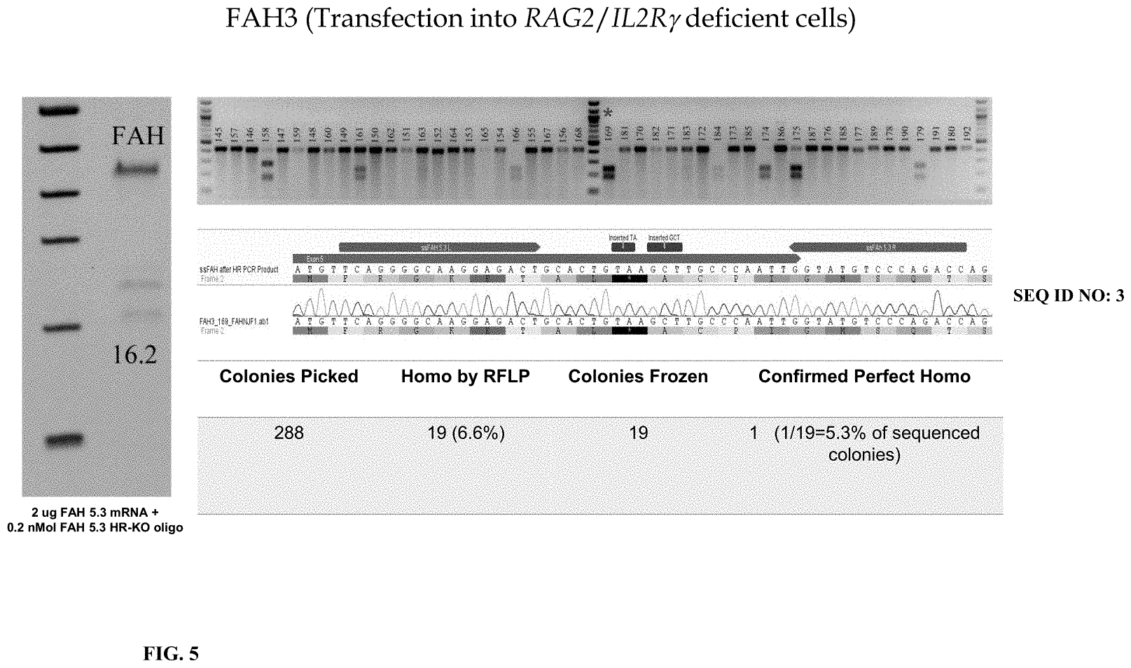

FIG. 5 shows the success of this strategy, with 5.3% of colonies sequence being positive for the augmentation of the FAH KO with the RG double KO. This triple FAH/IL2Rg/RAG2 KO is referred to as “FRG-KO”.

### Example 3

### Build and Test RegRG-KO and RegFRG-KO In Vitro

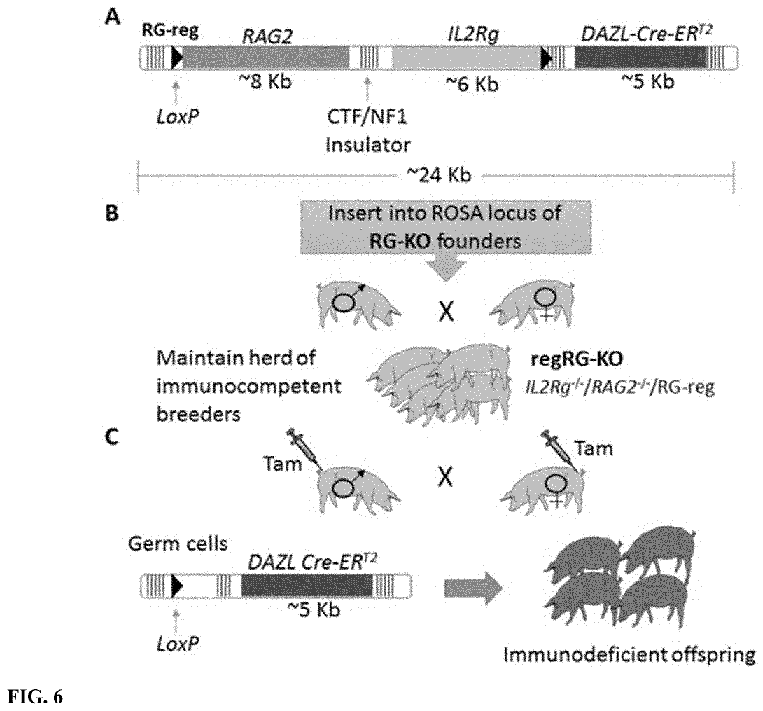

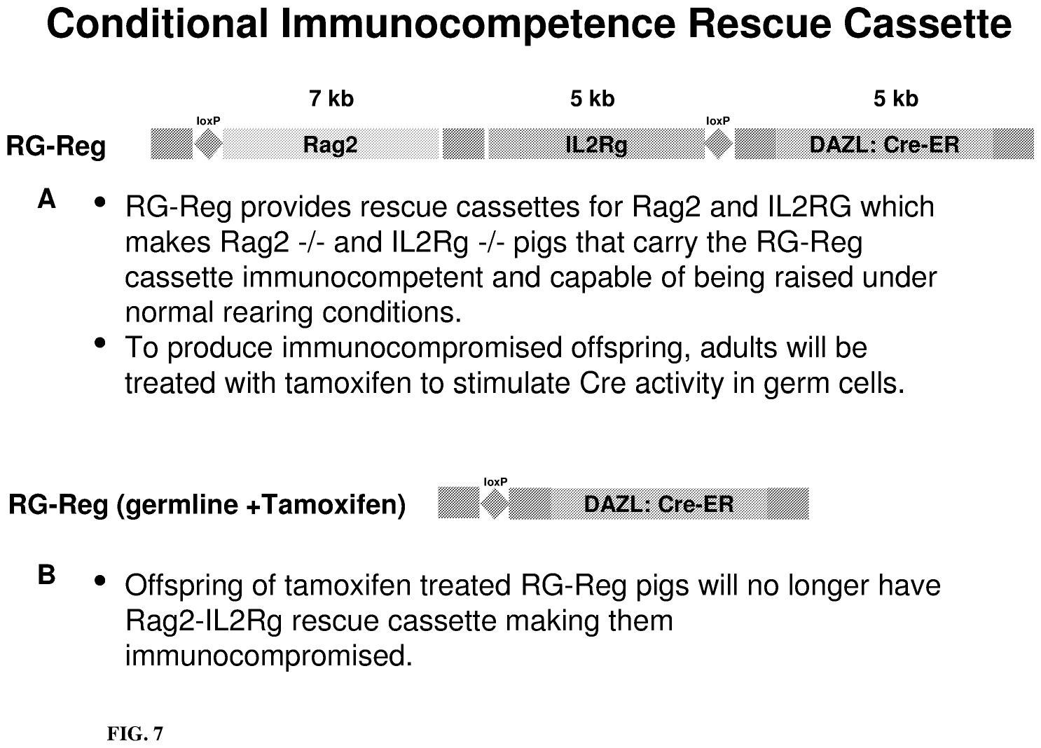

The overarching design of the inducible rescue cassette, RG-reg and FRG-reg, is shown in FIG. 6. In the embodiment shown, the cassette comprises three principle components, RAG2 and IL2Rg each driven by their native promoters and Cre-ERT224 driven by the DAZL promoter, FIG. 7. Each component is developed and tested individually prior to assembly of the entire cassette. Those of skill in the art will appreciate that when the background of the animal is FRG-KO, the cassette will have four principle components, RAG2, IL2Rg, FAH each driven by their native promoters and Cre-ER^{T224} driven by the DAZL promoter.

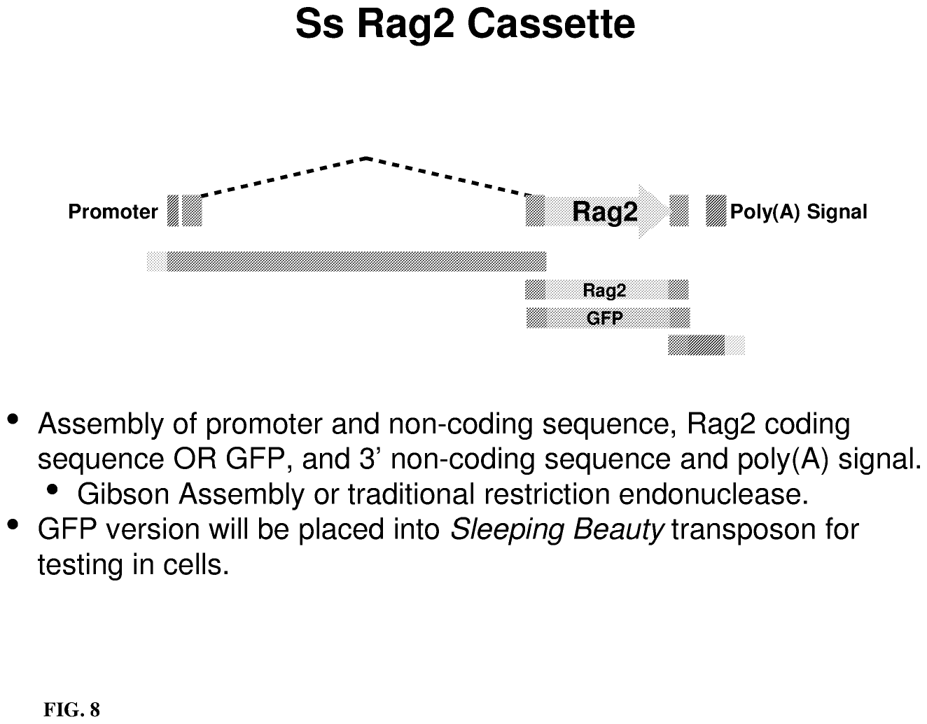

Component 1 consists of the porcine RAG2 gene and regulatory elements, FIG. 8. The entire genomic sequence of the gene as annotated in Ensembl is 5.93 Kb. Based on the work of Kishi et. al., ˜86 bp upstream of the transcription start site is sufficient for lymphocyte specific expression²⁵. The inventors rational design further looks to incorporate upstream sequences with known transcription factor binding sites, and is estimated the entire promoter sequence will be ˜1 kb. In addition, 3′ of the gene is extended to ensure incorporation of the 3′ UTR and polyadenylation signal, estimated to extend ˜1 kb downstream of the termination codon. The ˜8 kb cassette is synthesized in a manner to enable assembly with the other two components after testing. The resulting construct is tested for expression in immortalized lymphocyte cell lines as well as off target cells including pig fibroblasts and LLC-PK1 cells by porcine specific qPCR and western blotting.

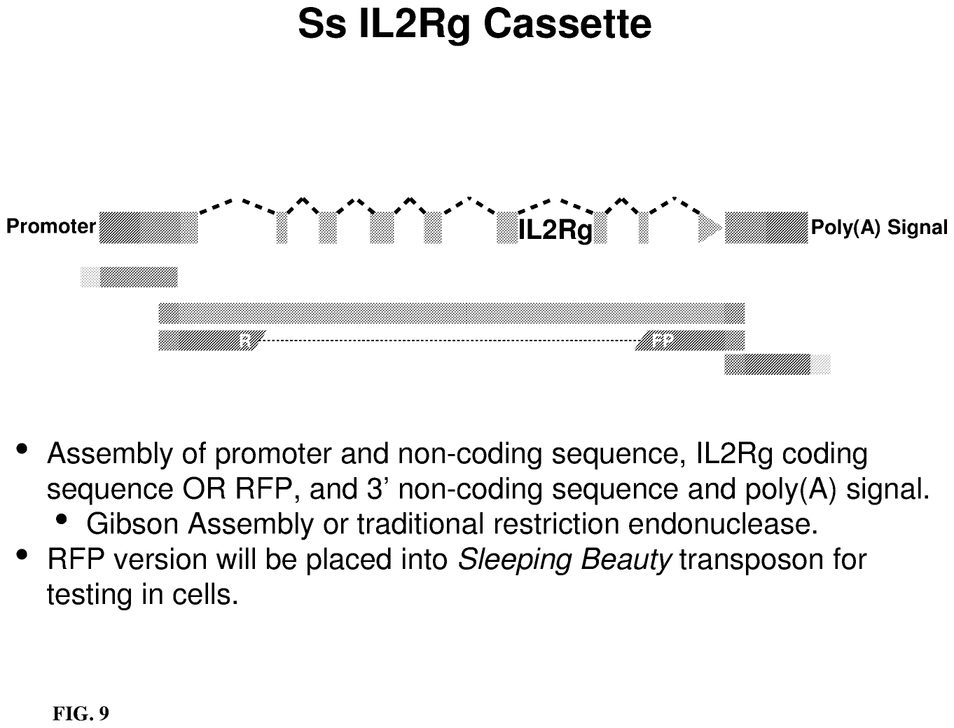

Component 2 similarly consists of the porcine IL2Rg gene with experimentally and bioinformatically designed regulatory elements, FIG. 9. Based on the work of Markiewicz et. al., the IL2Rg promoter consists of at least 1053 bp of 5′ promoter sequence²⁶. With an additional 1 kb 3′ sequence, the entire IL2Rg component is ˜6 kb. Component 2 is tested for expression in the same manner as component 1.

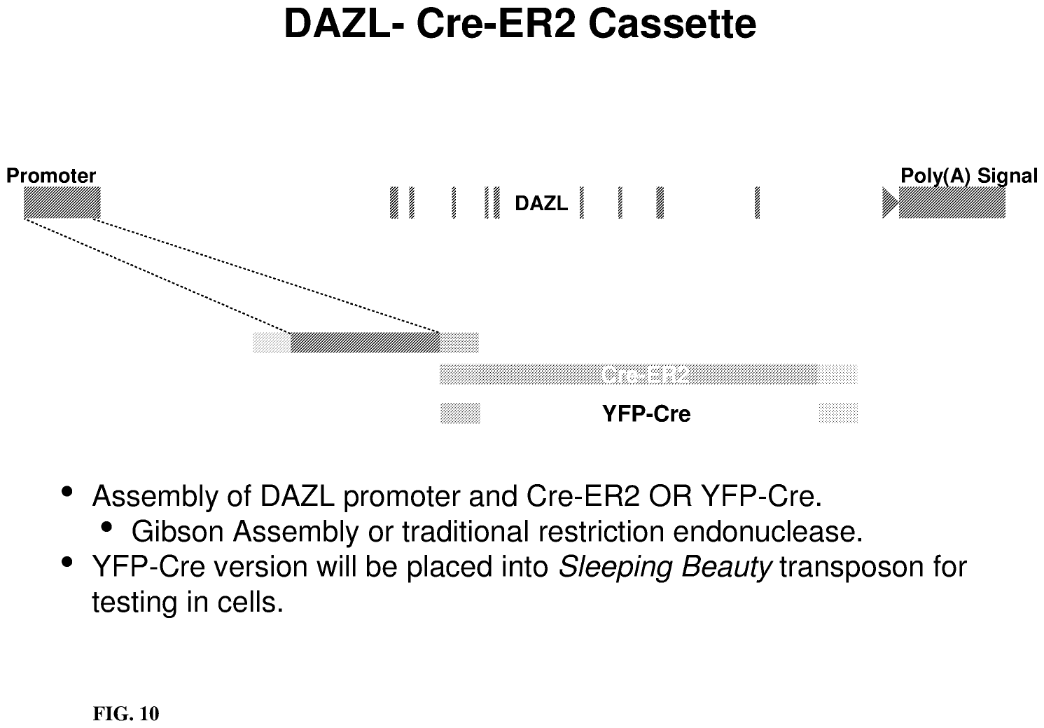

Component 3 is the driver of the Tamoxifen regulated “off switch” for components 1 and 2, FIG. 10. Briefly, ˜1.7 kb of upstream sequence and the non-transcribed portion of exon 1 from the porcine DAZL gene is cloned 5′ of the CreER^{T2} cDNA. This promoter region in mice directs EGFP expression exclusively to male and female germ cells²⁷. For in vitro testing, the DAZL-CreER^{T2} cassette is co-transfected with a Cre-activated LoxP-mCherrySTOP-LoxP-EGFP cassette previously validated in porcine cells²⁸. Cassettes are introduced into off target fibroblasts and LLC-PK1 in the presence or absence of Tamoxifen. Additionally, the construct is tested in isolated porcine germline stem cells^{29,30}. Knowing that CpG methylation plays a critical role in regulation of DAZL expression³¹, the inventors do expect leaky expression of CreER^{T2} in vitro as the plasmids will not be methylated; however, once integrated, the promoter methylation state is expected to reflect that of the endogenous gene.

Of course, for the FRG rescue cassette, FAH with its regulatory elements is also included along with IL2Rg and RAG2. Those of skill in the art will appreciate that when the animals are used as disease models, the methods used for their rescue allows for further research of individual conditions in animals that are otherwise extremely immunocompromised. Therefore, in some instances, the rescue cassette may not rescue the animal for all the genetic edits. For example, the FRG-KO animals may be rescued by cassette having one, two or all three genes restored on the cassette. In some cases, only IL2Rg and FAH may be present on the cassette. In other embodiments, only FAH and RAG2.

After demonstrating satisfactory expression in vitro, the components are assembled into a single vector by Gibson Assembly or any other method known to those of skill in the art. The final vector may include CTF/NF1 insulator elements to restrict interference of enhancer/repressor activates of each component³². Unidirectional LoxP sites will flank the RAG2 and IL2Rg and/or FAH genes to enable one-way Tamoxifen induced excision in germ cells (FIG. 4). Finally, the entire cassette is introduced/integrated into the porcine safe harbor locus ROSA of RG-KO fibroblasts locus using techniques such as PITCh or HITI as described herein^{33,34}. It should be noted, as those of skill in the art recognize, Tam-Cre is not the only inducible system that could be used in this way. For example, Tet or other systems as discussed above for “inducible systems” could be used.

### Example 4

### Produce Founder RegRG-KO and RegFRG Animals for Herd Expansion and Prototyping.

SCNT or embryo injection is used to generate regRG-KO and regFRG fibroblasts or zygotes. It is expected to observe normal levels of T, B and NK cells in regRG-KO and regFRG-KO pigs. Furthermore, after breeding and cryopreservation with regRG-KO or regFRG-KO semen, boars are pulsed with Tamoxifen and semen collected at regular intervals before and after. A three-primer assay is utilized to determine the extent of excision in the male germline. Timing and dosage of Tamoxifen will be further evaluated in subsequent generations of male and female regRG-KO and regFRG-KO.

Several quality control steps are built into components 1 and 2 above to ensure the highest probability of success for regRG-KO and regFRG-animals. It is postulated that in vitro tests may not accurately reflect performance in vivo. Hence, the performance is carefully assessed in animals empirically. A number of CreER^{T2} lines have been developed in rodents and excision of the recombinase elements within target cells in some cases is mosaic³⁵. However, the majority of these studies utilize only a single pulse of Tamoxifen to trace a given cell type. If required, Tamoxifen treatment in pigs can extend for months. If excision is limited to 80% of germ cells, it can still expect that 64% of resulting offspring will be immunodeficient after in-cross of two regRG-KO pigs; a 10-fold improvement over in-cross of heterozygous animals (Table 1).

Of course, those of skill in the art will appreciate that unless the animal is pulsed with tamoxifen, its germ line cells may continue to comprise the rescue cassette, in which case its progeny will continue to carry the edited KO genes but will be phenotypically wildtype.

### Example 5

### Production of Humanized Tissues and Organs in Immunodeficient Swine

Complementation of host RG-KO or the FRG-KO cell or embryos with totipotent or pluripotent cells is used to produce organs or tissues from donor cells. A non-limiting example of suites of genes responsible for organ and tissue development is provided in Table 2. A combination of knockouts of any of the genes identified in Table 2 creates a niche in the host cell or embryo for the complementation of the organs/tissues identified in Table 2 by human donor cells in a host background that is immune incompetent and cannot not launch an immune response against the human cells. Complementation of the host RG-KO or the FRG-KO cell or embryo with un-edited totipotent or pluripotent cells is used to produce the identified organs or tissues from the same lineage as the donor cells, e.g., if human cells, including stem cells, such as IPSC are used, the complemented organs or tissue would be humanized. Of course, it should be appreciated that the knockout of genes responsible for the development of any organ or tissue in the host cell or embryo can be accomplished by multiplex gene editing (see, for example, WO2015/168125, hereby incorporated by reference in its entirety) or serial edits or both. Those of skill understand that such organs or tissues could be customized for any individual in need. Thus, not only would the swine host, not recognize the donor cells as foreign, the donor, upon introduction or transplant of the complemented tissues would recognize them as “self”.

In addition, in some embodiments, once a desired background has been identified, a population of pigs edited to have such background may be bred to form a stable, well studied population for further experiments. For example, by maintaining a breeding herd of regRG-KO, it is possible to further augment the genetic edits and introduced into the animals to provides niches for organs requiring different genetic knockouts. In these examples, the rescue cassette could also be augmented in the primary cell or embryo such that such that 10 of genes could be knocked out or converted to disease causing alleles while the genes expressed from the rescue cassette could also be augmented to mirror the genes edited in the genome. Such augmentation, both in the genome and of the rescue cassette is accomplished by multiplex or serial editing of the genome and PITCh and HITI techniques to insert rescue genes into the cassette as described above. In this way a healthy population of host animals is created so as to provide populations of embryos for successful complementation of any organ or tissue desired and the organ or tissue could be maintained in an immunodeficient background allowing for their development in a niche that also includes an immune system derived from the same cells used for complementation. A non-comprehensive, abbreviated list of some genes responsible for the development of particular organs and tissues is provided in Table 2.

TABLE 2
TABLE 2
Carrier and Host Genotype - Complementation Product

| | DONOR | |
|---|---|---|
| HOST (Blastocyst, | (Blastocyst, | |
| Embryo, Zygote, | Embryo, | |
| cell | Zygote, cell) | FUNCTION |
| NURR1^{-/-}/LMX1A^{-/-}/PITX3^{-/-} | WT | Production of |
| LMX1A^{-/-}ITX3^{-/-} | | Dopamine |
| NURR1^{-/-}/LMX1A^{-/-}, | | neurons |
| NURR1^{-/-}/PITX3^{-/-} | | |
| NURR1^{-/-} | | |
| LMX1A^{-/-} | | |
| PITX3^{-/-} | | |
| OLIG^{-/-}/OLIG2^{-/-} | WT | Production of |
| OLIG^{-/-} | | Oligodendroglia |
| OLIG2^{-/-} | | |
| RAG2^{-/-}/IL2rg^{-/-}/ | WT | Production of |
| C-KIT^{-/-}/ETV2^{-/-} | | Young Blood |
| RAG2^{-/-}/IL2rg^{-/-}/C-KIT^{-/-} | | |
| RAG2^{-/-}/IL2rg^{-/-}/ETV2^{-/-} | | |
| IL2rg^{-/-}/C-KIT^{-/-}/ETV2^{-/-} | | |
| RAG2^{-/-}/IL2rg^{-/-} | | |
| RAG2^{-/-}/C-KIT^{-/-} | | |
| RAG2^{-/-}/ETV2^{-/-} | | |
| IL2rg^{-/-}/C-KIT^{-/-} | | |
| IL2rg^{-/-}/ETV2^{-/-} | | |
| C-KIT^{-/-}/ETV2^{-/-} | | |
| RAG2^{-/-}/IL2rg^{-/-}, | | |
| C-KIT^{-/-}/ETV2^{-/-} | | |
| C-KIT^{-/-} | | |
| ETV2^{-/-} | | |
| RUNX1^{-/-}/KIT^{-/-}/FLK1^{-/-} | WT | Hematopoietic |
| RUNX1^{-/-}/FLK1^{-/-} | | Cells Skin |
| RUNX1^{-/-}/KIT^{-/-} | | Repair |
| KIT^{-/-}/FLK1^{-/} | | |
| RUNX1^{-/-} | | |
| KIT^{-/-} | | |
| FLK1^{-/-} | | |
| HLA^{+/+}/TCR^{-/-}/HLA-A^{-/-} | CAR^{+/+} T; | Target Cancer |
| IL2Rγ^{-/-}/RAG1^{-/-}/RAG2^{-/-} | HLA^{+/+}/TCR^{-/-}/ | |
| (RAG 1/2) | HLA-A^{-/-} | |
| IL2Rγ^{-/-}/RAG1^{-/-}/ | | |
| IL2Rγ^{-/-}/RAG2^{-/-} | | |
| c-MPL^{-/-}, G6bB^{-/-}, SHP1^{-/-}, | HLA | Platelet |
| HSP2^{-/-} | classI^{neg} | Production |
| c-MPL^{-/-}/G6bB^{-/-}/ | iPSC | |
| SHP1^{-/-}/HSP2^{-/-} | WT | |
| c-MPL^{-/-}/G6bB^{-/-}/SHP1^{-/-} | HLA | |
| c-MPL^{-/-}/G6bB^{-/-}/HSP2^{-/-} | classI^{neg} | |
| c-MPL^{-/-}/SHP1^{-/-}/HSP2^{-/-} | | |
| G6bB^{-/-}/ SHP1^{-/-}/HSP2^{-/-} | | |
| c-MPL^{-/-}/G6bB^{-/-} | | |
| c-MPL^{-/-}/SHP1^{-/-} | | |
| c-MPL^{-/-}/HSP2^{-/-} | | |
| G6bB^{-/-}/ SHP1^{-/-} | | |
| G6bB^{-/-}/HSP2^{-/-} | | |
| c-MPL^{-/-} | | |
| G6bB^{-/-} | | |
| SHP1^{-/-} | | |
| HSP2^{-/-} | | |
| ETV2^{-/-} | WT | Blood Vessels |
| | | Vasculature |
| | | Repair |
| NKX2-5^{-/-}/HANDII^{-/-}/TBX5^{-/-} | WT | Myocardiocytes |
| NKX2-5^{-/-}/HANDII^{-/-} | | Restoring |
| HANDII^{-/-} | | Cardiac |
| NKX2-5^{-/-} | | Function |
| TBX5^{-/-} | | |
| MYF5^{-/-}/MYOD^{-/-}/MRF4^{-/-} | WT | Skeletal Muscle |
| MYF5^{-/-}/MYOD^{-/-} | | |
| MYF5^{-/-}/MRF4^{-/-} | | |
| MYOD^{-/-}/MRF4^{-/-} | | |
| MYF5^{-/-} | | |
| MYOD^{-/-} | | |
| MRF4^{-/-} | | |
| PAX3^{-/-} | | |
| HHEX^{-/-}/Ubc^{-/-} | WT | Liver/Hepatocytes |
| HHEX^{-/-} | | |
| Ubc^{-/-} | | |
| FAH | | |
| Pdx1^{-/-}/Etv2^{-/-} | WT | Pancreas |
| Pdx1^{-/-} | | Transplants and |
| Etv2^{-/-} | | Insulin |
| | | Production |
| Nkx2.1^{-/-}/Sox2^{-/-}/ | WT | Lung/Pulmonary |
| Id2^{-/-}/Tbx4^{-/-} | | Tissue |
| Nkx2.1^{-/-}/Sox2^{-/-}/Id2^{-/-} | | |
| Nkx2.1^{-/-}/Sox2^{-/-}/Tbx4^{-/-} | | |
| Nkx2.1^{-/-}/Id2^{-/-}/Tbx4^{-/-} | | |
| Sox2^{-/-}/Id2^{-/-}/Tbx4^{-/-} | | |
| Nkx2.1^{-/-}/Sox2^{-/-} | | |
| Nkx2.1^{-/-}/Id2^{-/-} | | |
| Nkx2.1^{-/-}/Tbx4^{-/-} | | |
| Sox2^{-/-}/Id2^{-/-} | | |
| Sox2^{-/-}/Tbx4^{-/-} | | |
| Id2^{-/-}/Tbx4^{-/-} | | |
| Nkx2.1^{-/-} | | |
| Sox2^{-/-}/Id2^{-/-} | | |
| Tbx4^{-/-} | | |
| Pax2^{-/-}/Pas8^{-/-} | WT | Kidney Renal |
| Pax2^{-/-} | | Function |
| Pax8^{-/-} | | |

While this invention has been described in conjunction with the various exemplary embodiments outlined above, various alternatives, modifications, variations, improvements and/or substantial equivalents, whether known or that are or may be presently unforeseen, may become apparent to those having at least ordinary skill in the art. Accordingly, the exemplary embodiments according to this disclosure, as set forth above, are intended to be illustrative not limiting. Various changes may be made without departing from the spirit and scope of the invention. Therefore, the invention is intended to embrace all known or later-developed alternatives, modifications, variations, improvements and/or substantial equivalents of these exemplary embodiments.

The following paragraphs enumerated consecutively from 1 through 45 provide for various additional aspects of the present invention. In one embodiment, in a first paragraph:

1. A rescue cassette comprising:

a germ-line specific promoter fused to an inducible recombinase;
one or more rescue genes wherein the rescue genes are homologs or orthologs to native genes found in livestock animals; and
wherein the genes in the cassette are under the control of their native promoter, wherein the cassette is configured for introgression into the genome of a primary cell or embryo of a livestock animal.

2. The cassette of paragraph 1, configured to excise the cassette in germ-line cells, upon induction of the recombinase in vivo.

3. The cassette of paragraphs 1-2, wherein the rescue genes are driven by their native promoters.

4. The cassette of paragraphs 1-3, wherein the cassette is introduced into a cell or embryo.

5. The cassette of paragraphs 1-4, further comprising a landing pad.

6. The cassette of paragraphs 1-5, wherein the cassette is augmented comprising introduction of one or more additional genes into the cassette.

7. The cassette of paragraphs 1-6, wherein the recombinase is induced by an estrogen receptor antagonist.

8. The cassette of paragraphs 1-7, wherein the estrogen receptor antagonist is tamoxifen.

9. The cassette of paragraphs 1-8, wherein the tissue specific promoter is a gametogenic promoter.

10. The cassette of paragraphs 1-9, wherein the gametogenic promoter is a DAZL promoter, a VASA promoter or a NANOS promoter.

11. A cell or embryo having introduced therein the cassette paragraphs 1-10.

12. An animal produced from the cell or embryo of paragraphs 1-11.

13. A cell or embryo having introduced therein the cassette of paragraphs 1-12, wherein the cell or embryo further has one or more homologs or orthologs of the genes contained in the cassette edited.

14. The cell or embryo of paragraphs 1-13, wherein the cassette is integrated into the genome at a safe harbor locus.

15. The cell or embryo of paragraphs 1-13, wherein the edits comprise knock-outs or conversions to a synthetic sequence or a disease allele.

16. The cell or embryo of paragraphs 1-13, wherein the rescue genes expressed from the cassette are from the same species as the edited genes.

17. The cell or embryo of paragraphs 1-13, wherein the edited genes comprise Interleukin 2 Receptor Subunit Gamma (IL2rg) and/or Recombination Activating 2 (RAG2) and/or Fumarylacetoacetate Hydrolase (FAH).

18. The cell or embryo of paragraphs 1-13, wherein the cell is cloned, or the embryo implanted in a surrogate mother.

19. A livestock animal produced from the cell of embryo of paragraphs 1-15.

20. A livestock animal comprising an edited genome and in its genome a rescue cassette including an inducible recombinase, wherein the rescue cassette is expressed in a majority of the cells of the animal and wherein one or more of the animals native genes are edited wherein the cassette expresses one or more rescue genes homologous or otholgous to the edited native genes, wherein the rescue cassette includes an inducible recombinase driven by a tissue specific promoter, wherein the tissue specific promoter is gamete-specific.

21. The livestock animal of paragraph 20, wherein the rescue cassette is integrated into a safe harbor locus of the animal's genome.

22. The livestock animal of paragraphs 20-21, wherein one or more of the edited genes comprise a niche for organ or tissue development.

23. The livestock animal of paragraphs 20-22, wherein the livestock animal has a wild-type phenotype.

24. The livestock animal of paragraphs 20-23, wherein the animal is porcine, bovine, caprine (goat or sheep).

25. The livestock animal of paragraphs 20-25, wherein, upon induction of the recombinase, the cassette is excised from the gametes of the animal.

26. An embryo derived from fertilization of a male game and a female gamete of paragraphs 20-25.

27. The embryo of paragraphs 1-26, further complemented by one or more pluripotent cells.

28. The embryo of paragraphs 1-27, wherein the pluripotent cells are human.

29. The embryo of paragraphs 1-26, wherein the embryo does not express IL2Rg and/or RAG2.

30. Progeny of a male and female animal of paragraph 20.

31. A method of making a livestock animal model of disease comprising:

editing one or more genes associated with a disease in a fibroblast or embryo of an animal;

integrating into the fibroblast or embryo genome a rescue cassette comprising:

one or more rescue genes homologous or orthologous to the edited genes;

an inducible recombinase under control of a tissue specific promoter;

wherein the tissue specific promoter is gamete specific;

inducing the recombinase, wherein the rescue cassette is excised from the gametogenic tissue;

wherein the gametes of the animal do not contain the rescue cassette;

wherein a female gamete is fertilized by a male gamete to provide an embryo;

wherein the embryo is gestated to an animal.

32. The method of paragraph 31, wherein the male and female gametes have the same genetic edits.

33. The method of paragraphs 31-32, wherein the male and female gametes have different genetic edits.

34. the method of paragraphs 31-33, wherein the gamete specific promoter comprises a DAZL promoter, a VASA promoter or a NANOS promoter.

35. The method of paragraphs 31-34, wherein the genetic edits introduce disease alleles into the genome.

36. The method of paragraphs 31-35, wherein the genetic edits result in knockouts of the genes.

37. The method of paragraphs 31-36, wherein the genetic edits introduce a niche for the development of organs or tissues.

38. The method of paragraphs 31-37, wherein pluripotent cells are introduced into the embryo to complement the niche.

39. The method of paragraphs 31-38, wherein the pluripotent cells are derived from a different species than the embryo.

40. The method of paragraphs 31-39, wherein the pluripotent cells are human.

41. The method of paragraphs 31-40, wherein the embryo is pig, goat, sheep or cow.

42. The method of paragraphs 31-41, wherein, before inducing, the embryo is further, modified, comprising editing one or more further genes and, the rescue cassette of the embryo is modified to introduce one or more homologs of the one or more further genes, wherein an animal is produced from the embryo, providing an F1 generation.

43. The method of paragraphs 31-42, wherein the recombinase is induced in a male and a female of the F1 generation and an embryo is produced from the induced F1 gametes, wherein the embryo is complemented with one or more pluripotent cells, wherein the pluripotent cells complement the niche of the edited genes of the F1 generation.

44. The method of paragraphs 31-43, wherein the edited genes comprise RAG2 and/or IL2Rg.

45. The method of paragraphs 31-44, wherein the further genes modified are FAH.

All patents, publications, and journal articles set forth herein are hereby incorporated by reference herein; in case of conflict, the instant specification is controlling.

While this invention has been described in conjunction with the various exemplary embodiments outlined above, various alternatives, modifications, variations, improvements, and/or substantial equivalents, whether known or that are or may be presently unforeseen, may become apparent to those having at least ordinary skill in the art. Accordingly, the exemplary embodiments according to this invention, as set forth above, are intended to be illustrative, not limiting. Various changes may be made without departing from the spirit and scope of the invention. Therefore, the invention is intended to embrace all known or later-developed alternatives, modifications, variations, improvements, and/or substantial equivalents of these exemplary embodiments.

### BIBLIOGRAPHY

1. 1Yamada, S. et al. Regenerative Therapy Prevents Heart Failure Progression in Dyssynchronous Nonischemic Narrow QRS Cardiomyopathy. J Am Heart Assoc 4, doi:10.1161/JAHA.114.001614 (2015).
2. 2Hickey, R. D. et al. Efficient production of Fah-null heterozygote pigs by chimeric adeno-associated virus-mediated gene knockout and somatic cell nuclear transfer. Hepatology 54, 1351-1359, doi:10.1002/hep.24490 (2011).
3. 3Bailey, A. M., Mendicino, M. & Au, P. An FDA perspective on preclinical development of cell-based regenerative medicine products. Nature biotechnology 32, 721-723, doi:10.1038/nbt.2971 (2014).
4. 4Kobayashi, T. et al. Generation of rat pancreas in mouse by interspecific blastocyst injection of pluripotent stem cells. Cell 142, 787-799, doi:10.1016/j.cell.2010.07.039 (2010).
5. 5Matsunari, H. et al. Blastocyst complementation generates exogenic pancreas in vivo in apancreatic cloned pigs. Proc Natl Acad Sci USA 110, 4557-4562, doi:10.1073/pnas.1222902110 (2013).
6. 6Wu, J. et al. Interspecies Chimerism with Mammalian Pluripotent Stem Cells. Cell 168, 473-486 e415, doi:10.1016/j.cell.2016.12.036 (2017).
7. 7Grompe, M. & Strom, S. Mice with human livers. Gastroenterology 145, 1209-1214, doi:10.1053/j.gastro.2013.09.009 (2013).
8. 8Shultz, L. D., Brehm, M. A., Garcia-Martinez, J. V. & Greiner, D. L. Humanized mice for immune system investigation: progress, promise and challenges. Nat Rev Immunol 12, 786-798, doi:10.1038/nri3311 (2012).
9. 9Hickey, R. D. et al. Curative ex vivo liver-directed gene therapy in a pig model of hereditary tyrosinemia type 1. Sci Transl Med 8, 349ra399, doi:10.1126/scitranslmed.aaf3838 (2016).
10. 10Zitvogel, L., Kepp, O. & Kroemer, G. Immune parameters affecting the efficacy of chemotherapeutic regimens. Nat Rev Clin Oncol 8, 151-160, doi:10.1038/nrclinonc.2010.223 (2011).
11. 11Illheana, E., Champiat, S. & Soria, J. C. [Immune-checkpoints: the new anti-cancer immunotherapies]. Bull Cancer 100, 601-610, doi:10.1684/bdc.2013.1771 (2013).
12. 12Pardoll, D. M. The blockade of immune checkpoints in cancer immunotherapy. Nat Rev Cancer 12, 252-264, doi:10.1038/nrc3239 (2012).
13. 13Fischbach, M. A., Bluestone, J. A. & Lim, W. A. Cell-based therapeutics: the next pillar of medicine. Sci Transl Med 5, 179ps177, doi:10.1126/scitranslmed.3005568 (2013).
14. 14June, C. H. Adoptive T cell therapy for cancer in the clinic. J Clin Invest 117, 1466-1476, doi:10.1172/JCI32446 (2007).
15. 15Rosenberg, S. A. Adoptive immunotherapy of cancer: accomplishments and prospects. Cancer Treat Rep 68, 233-255 (1984).
16. 16Restifo, N. P., Dudley, M. E. & Rosenberg, S. A. Adoptive immunotherapy for cancer: harnessing the T cell response. Nat Rev Immunol 12, 269-281, doi:10.1038/nri3191 (2012).
17. 17Cibelli, J. et al. Strategies for improving animal models for regenerative medicine. Cell Stem Cell 12, 271-274, doi:10.1016/j.stem.2013.01.004 (2013).
18. 18Butler, J. E. et al. The piglet as a model for B cell and immune system development. Vet Immunol Immunopathol 128, 147-170, doi:10.1016/j.vetimm.2008.10.321 (2009).
19. 19Suzuki, S. et al. 112rg gene-targeted severe combined immunodeficiency pigs. Cell Stem Cell 10, 753-758, doi:10.1016/j.stem.2012.04.021 (2012).
20. 20Watanabe, M. et al. Generation of interleukin-2 receptor gamma gene knockout pigs from somatic cells genetically modified by zinc finger nuclease-encoding mRNA. PLoS One 8, e76478, doi:10.1371/journal.pone.0076478 (2013).
21. 21Lee, K. et al. Engraftment of human iPS cells and allogeneic porcine cells into pigs with inactivated RAG2 and accompanying severe combined immunodeficiency. Proc Natl Acad Sci USA 111, 7260-7265, doi:10.1073/pnas.1406376111 (2014).
22. 22Azuma, H. et al. Robust expansion of human hepatocytes in Fah−/−/Rag2−/−/Il2rg−/− mice. Nature biotechnology 25, 903-910 (2007).
23. 23Tan, W. et al. Efficient nonmeiotic allele introgression in livestock using custom endonucleases. Proc Natl Acad Sci USA 110, 16526-16531, doi:10.1073/pnas.1310478110 (2013).
24. 24Feil, R., Wagner, J., Metzger, D. & Chambon, P. Regulation of Cre recombinase activity by mutated estrogen receptor ligand-binding domains. Biochemical and biophysical research communications 237, 752-757, doi:10.1006/bbrc.1997.7124 (1997).
25. 25Kishi, H. et al. Lineage-specific regulation of the murine RAG-2 promoter: GATA-3 in T cells and Pax-5 in B cells. Blood 95, 3845-3852 (2000).
26. 26Markiewicz, S. et al. Tissue-specific activity of the gammac chain gene promoter depends upon an Ets binding site and is regulated by GA-binding protein. J Biol Chem 271, 14849-14855 (1996).
27. 27Nicholas, C. R. et al. Characterization of a Dazl-GFP germ cell-specific reporter. Genesis 47, 74-84, doi:10.1002/dvg.20460 (2009).
28. 28Clark, K. J. et al. Enzymatic engineering of the porcine genome with transposons and recombinases. BMC Biotechnol 7, 42, doi:10.1186/1472-6750-7-42 (2007).
29. 29Honaramooz, A., Megee, S. O. & Dobrinski, I. Germ cell transplantation in pigs. Biology of reproduction 66, 21-28 (2002).
30. 30Zeng, W. et al. Viral transduction of male germline stem cells results in transgene transmission after germ cell transplantation in pigs. Biology of reproduction 88, 27, doi:10.1095/biolreprod.112.104422 (2013).
31. 31Li, B. et al. Altered DNA Methylation Patterns of the H19 Differentially Methylated Region and the DAZL Gene Promoter Are Associated with Defective Human Sperm. PLoS One 8, e71215, doi:10.1371/journal.pone.0071215 (2013).
32. 32Gaussin, A. et al. CTF/NF1 transcription factors act as potent genetic insulators for integrating gene transfer vectors. Gene Ther 19, 15-24, doi:http://www.nature.com/gt/journal/v19/n 1/suppinfo/gt201170s1.html (2012).
33. 33Nakade, S. et al. Microhomology-mediated end-joining-dependent integration of donor DNA in cells and animals using TALENs and CRISPR/Cas9. Nature communications 5, 5560, doi:10.1038/ncomms6560 (2014).
34. 34Suzuki, K. et al. In vivo genome editing via CRISPR/Cas9 mediated homology-independent targeted integration. Nature 540, 144-149, doi:10.1038/nature20565 (2016).
35. 35Feil, S., Valtcheva, N. & Feil, R. Inducible Cre mice. Methods Mol Biol 530, 343-363, doi:10.1007/978-1-59745-471-1_18 (2009).

1. A rescue cassette comprising:
one or more rescue genes under the control of their native promoter that are homologs to native genes in a pig that have been knocked out; and
a germline specific promoter fused to an inducible recombinase wherein upon induction of the inducible recombinase results in excision of the rescue cassette in gametes of the pig but not in non-gamete cells of the pig for breeding of an immunocompetent pig for rapid propagation.

2. The cassette of claim 1, configured to excise the cassette in germ-line cells, upon induction of the recombinase in vivo by exposure to tamoxifen.

3. (canceled)

4. The cassette of claim 1, wherein the cassette is introduced into a cell or an embryo.

5. The cassette of claim 1, further comprising a landing pad.

6. The cassette of claim 1, wherein the cassette is augmented comprising introduction of one or more additional genes into the cassette.

7-8. (canceled)

9. The cassette of claim 1, wherein the germ-line specific promoter is a gametogenic promoter.

10. (canceled)

11. A cell or embryo having introduced therein the cassette claim of 1.

12. An animal produced from the cell or embryo of claim 11.

13. A cell or embryo having introduced therein the cassette of claim 1, wherein the cell or embryo has in its genome one or more homologs or orthologs of the rescue genes contained in the cassette, wherein the one or more homologs or orthologs are edited.

14. The cell or embryo of claim 13, wherein the cassette is integrated into the genome at a safe harbor locus.

15. The cell or embryo of claim 13, wherein the edited genes comprise knock-outs or conversions to a synthetic sequence or disease alleles.

16. (canceled)

17. The pig of claim 12, wherein the one or more rescue genes comprise Interleukin 2 Receptor Subunit Gamma (IL2rg), Recombination Activating 2 (RAG2), or Fumarylacetoacetate Hydrolase (FAH).

18-30. (canceled)

31. A method of making a pig model of disease comprising:
editing one or more genes associated with a disease in a fibroblast or embryo of an animal;
integrating into the fibroblast or embryo genome a rescue cassette comprising:
one or more rescue genes of the edited genes;
an inducible recombinase under control of a tissue specific promoter;
wherein the tissue specific promoter is gamete specific;
inducing the recombinase, wherein the rescue cassette is excised from the gametogenic tissue;
wherein the gametes of the animal do not contain the rescue cassette;
wherein a female gamete is fertilized by a male gamete to provide an embryo;
wherein the embryo is gestated to an animal.

32-35. (canceled)

36. The method of claim 31, wherein the genetic edits result in knockouts of the genes.

37. The method of claim 36, wherein the genetic edits introduce a niche for the development of organs or tissues.

38. The method of claim 31, wherein pluripotent cells are introduced into the embryo to complement the niche.

39-45. (canceled)

46. The pig of claim 12, wherein the inducible recombinase is a Cre recombinase and wherein induction comprises exposure of the pig to tamoxifen.

47. A breeding herd comprising a plurality of pigs of claim 12.

48. A pig produced from a pig cell or pig embryo comprising a rescue cassette integrated into the genome of the pig cell or swine embryo or pig embryo, wherein the rescue cassette comprises: one or more rescue genes under the control of their native promoter that are homologs to native genes in the pig that have been knocked out; and a germline specific promoter fused to an inducible recombinase wherein upon induction of the inducible recombinase results in excision of the rescue cassette in gametes of the pig, but not in non-gamete cells of the pig for breeding of an immunocompetent pig for rapid propagation.

49. A breeding herd comprising a plurality of pigs of claim 48.
